(19) United States
(12) Patent Application Publication
(10) Pub. No.: US 2022/0406404 A1
(43) Pub. Date: Dec. 22, 2022

(54) ADVERSARIAL FRAMEWORK FOR MOLECULAR CONFORMATION SPACE MODELING IN INTERNAL COORDINATES

(71) Applicant: Insilico Medicine IP Limited, Hong Kong (HK)

(72) Inventors: Maksim Kuznetsov, Moscow (RU); Fedor Ryabov, Korolev (RU); Daniil Polykovskiy, Moscow (RU); Artur Kadurin, Taipei City (TW); Aleksandrs Zavoronkovs, Pak Shek Kok (HK)

(21) Appl. No.: 17/835,167

(22) Filed: Jun. 8, 2022

Related U.S. Application Data
(60) Provisional application No. 63/208,904, filed on Jun. 9, 2021.

(51) Int. Cl. G16B 15/30; G16C 10/00

(52) U.S. Cl. CPC G16B 15/30; G16C 10/00

(57) ABSTRACT

A computer-implemented method for a generative adversarial approach for conformational space modeling of molecules is provided. The method can include obtaining molecule graph data for a molecule and inputting the molecule graph data into a machine learning platform. The machine learning platform can include architecture of a molecular graph generator, conformation discriminator, stochastic encoder, and latent variables discriminator. The method can include generating a plurality of conformations for the molecule with the machine learning platform. The plurality of conformations are specific to the molecule. Each conformation can have internal coordinates defining positions of atoms of the molecule. At least one conformation for the molecule can be selected based on at least one parameter related to molecular conformations. A report can be prepared that includes the selected at least one conformation for the molecule.

### CROSS-REFERENCE TO RELATED APPLICATIONS

This patent application claims priority to U.S. Provisional Application No. 63/208,904 filed Jun. 9, 2021, which provisional is incorporated herein by specific reference in its entirety.

### BACKGROUND

### Field

The present disclosure relates to a novel generative model for molecular conformation generation built upon the combination of a Generative Adversarial Network and Adversarial Autoencoder.

### Description of Related Art

Previously, there have been advances in machine learning approaches for solving fundamental problems of drug discovery (Chen et al. [2018]; Vamathevan et al. [2019]), drug candidate generation (Gomez-Bombarelli et al. [2018]; Zhavoronkov et al. [2019], Shayakhmetov et al. [2020]), chemical and biological properties prediction of small molecules (Wu et al. [2017]; Gilmer et al. [2017]), synthesis planning (Segler et al. [2018]), drug-target interaction prediction (Chen et al. [2018]; Kao et al. [2021]), and others. Still, the majority of approaches rely only on the 2D structural representation of chemical compounds, thus missing important spatial information. In the real world, each molecule takes the form of a 3D conformations set having a plurality of different 3D conformations. Identifying the set of probable conformations, defined as a conformation space, is useful for several challenging drug discovery tasks, including protein folding (Senior et al. [2020], Jumper et al. [2021]; Ingraham et al. [2019]) and virtual screening (van Hilten et al. [2019]). However, each molecule exists as a 3D arrangement of atoms for its conformation. Identifying the likelihood of conformations and conformation sampling is useful for drug discovery tasks.

Methodologies can evaluate molecular conformation space experimentally or computationally. The experimental methods, such as X-ray crystallography (Blundell et al. [2002]) or Nuclear Magnetic Resonance (Pellecchia et al. [2008]), involve time and cost-consuming measurement procedures. Moreover, experimental methods are limited to specific physical states, such as solid phase, and typically capture only a single most probable conformation, whereas some drug discovery tasks require information about the distribution of conformations. The computational methods of Molecular Dynamics (De Vivo et al. [2016]) rely on numerical modeling of interatomic interactions and quantum effects. These methods differ from each other in accuracy and speed—the most precise DFT (Mardirossian [2017]) methods are computationally extensive and require significantly more time to run in comparison to approximate force-field algorithms (Halgren [1999]). A new learning-based family of methods emerged recently (Dral [2020]), aiming both at the accuracy of ab initio calculations and the computational cost comparable to the force-field approach.

Deep learning techniques, which aim both at high accuracy and low inference time, show promising molecular conformation space modeling results. The recent works show the promising results of neural networks application in the problem of conformation space modeling. Several generative approaches represent molecular compounds in the Cartesian coordinates (Mansimov et al. [2019]) and the Euclidean distance geometries (EDG) (Xu et al. [2021a, b]; Simm et al. [2020]). Furthermore, SchNet (Schutt et al. [2018]) model and ANI (Smith et al. [2017]) succeeded in predicting the conformational energy landscape, allowing to perform neural-guided molecular dynamics. Despite extensive research and a growing number of publications, neural conformation modeling still contains several open problems and limitations.

Most current works evaluate the models only by conformation space coverage and diversity metrics, completely ignoring the physical plausibility of generated conformations. Despite high scores on the mentioned metrics, the learned models can still produce physically unrealistic conformations with incorrect geometry. For this reason, some metric that accounts for conformation energy can be used to evaluate the plausibility of generated conformations properly.

The training and evaluation of models are performed only on unconditional distributional learning tasks. For instance, the current architectures unconditionally generate all possible samples from the conformation space. Nevertheless, the actual drug discovery problems (Kombo et al. [2013]; Mason et al. [2001]) require the search of the best conformation with specific properties in conformation space. Therefore, comparing generation approaches on setups with external conditions imposed on conformations could be useful.

The quality of models strongly depends on conformation parameterization. The models that work directly with Cartesian coordinates suffer from the lack of rotation, translation, and reflection invariance. Approaches based on modeling interatomic distances may require additional techniques to satisfy triangle inequalities between all triples of atoms. Another alternative is to use internal coordinates, which cannot easily model cyclic and flat structures. Overall, existing models concentrate only on one parametrization technique, limiting their quality.

Molecular conformation space modeling has a long history of theoretical and applied research. The conformation space can be modeled from the first principles with quantum and classical algorithms. These algorithms evaluate the physical forces between atoms and produce energy with respect to atomic positions. The examples of these algorithms are ab initio methods and DFT (Mardirossian et al., [2017]). The most probable conformations are then sampled with molecular dynamics. These methods are accurate, but computationally too expensive for massive drug design tasks. The cheapest alternative is to iterate over predefined 3D structures of each molecular fragment to generate a combinatorial space of possible conformations (Cole et al. [2018]). Another alternative is to approximate interatomic interactions by rule-based force fields (Halgren [1999]) to evaluate the energy of conformations cheaply.

In recent years, multiple neural methods for molecular conformation modeling were developed. SchNet model (Schutt et al. [2018]) demonstrated high quality in molecular energy prediction. Following traditional approach, SchNet was adapted (Westermayr et al. [2020]) to perform the molecular dynamics on small molecules. The idea of learning the atomic gradient field for molecular dynamics was extended (Shi et al. [2021]), where it was proposed to learn the conformation space with a score-based generative model.

The iterative approach for conformation generation is also shared by models based on solving the Euclidean distance geometry problem (Liberti et al. [2012]). GraphDG (Simm et al., [2020]) is a variational autoencoder that models the distribution over distances. After a pair-wise distance matrix is generated, the EDG algorithm reconstructs Euclidean coordinates of a conformation from the pairwise distances. CGCF (Xu et al. [2021a]) model was proposed to learn the distribution over inter-atomic distances of molecules—the model generates a pairwise distance matrix, then EDG algorithm reconstructs 3D coordinates, and, optionally, the SchNet model refines the result. An autoencoder-based architecture ConfVAE proposed by (Xu et al. [2021b]) incorporates the EDG algorithm into the computational graph of the training procedure.

Several approaches for direct conformation sampling were developed to overcome the time-consuming iterative optimization process used in neural molecular dynamics and EDG-based models. Simm (Simm et al. [2020]) proposed a reinforcement-learning framework for conformation construction in Cartesian coordinates. GeoMol model (Ganea et al. [2021]) extended the idea of internal coordinates modeling and efficiently combined it with predicting the 3D structure of the atomic neighborhood.

Thus, there is a need for a technology that can be used for molecular conformation space modeling in internal coordinate in order to overcome the foregoing limitations in the state-of-the-art.

### SUMMARY

In some embodiments, a computer-implemented method for a generative adversarial approach for conformational space modeling of molecules is provided. The method can be performed with a computing system, such as described herein. The method can include obtaining molecule graph data for a molecule and inputting the molecule graph data into a machine learning platform. The machine learning platform can include architecture of a molecular graph generator, conformation discriminator, stochastic encoder, and latent variables discriminator. The method can include generating a plurality of conformations for the molecule with the machine learning platform. The plurality of conformations are specific to the molecule. Each conformation can have internal coordinates defining positions of atoms of the molecule. At least one conformation for the molecule can be selected based on at least one parameter related to molecular conformations. A report can be prepared that includes the selected at least one conformation for the molecule. The machine learning platform can predict lengths for each molecular graph bond of the molecule for each conformation. For example, at least one parameter that is related to molecular conformations can include an energy of each conformation. As such, the method can include providing at least one selected conformation of the molecule that has a lower energy compared to other generated conformations of the molecule. Also, the report can include a conformation space that is comprised of a plurality of overlaid selected conformations for the molecule.

In some embodiments, the method can include inputting molecule graph data of the molecule and a set of latent vectors into a generator and outputting a conformation of the molecule as a sequence of internal coordinates. Real conformations can be distinguished from generated conformations with predicted energy differences. The conformations can be mapped into latent space. The latent space can be conformed to be similar to a prior distribution (e.g., of conformations).

In some embodiments, the method can include a conformation generation protocol. The conformation generation protocol can include generating internal coordinates of a conformation from the molecule graph data and noise. The bond lengths and a bond-wise loss function weight can be predicted. The internal coordinates can be converted to Cartesian coordinates for the conformation. The method can include computing the Cartesian coordinates for unit direction and unit normal vectors and modulating bond length of a conformation to the predicted bond lengths.

In some embodiments, the method can include representing the molecular graph by nodes and edge feature sets and extending the molecular graph with auxiliary nodes and edges to make a proposed generative model. Virtual edges can be introduced between second, third, and/or fourth neighboring nodes. Each node can be set to include a description of: atom type, charge, and chiral tag. Each edge feature can be set to include a first graph subset that has chemical bond type and bond stereochemistry. Each edge feature can be set to include a second graph subset that has a spanning tree traversal process and having defining edge features to be in the spanning tree and information regarding whether a source node appears earlier in the spanning tree traversal process than a destination node.

In some embodiments, the method can include estimating one or more of the following conformation properties for each generated molecule: asphericity, eccentricity, inertial shape factor, two normalized principal moments ratios, three principal moments of inertia, gyration radius or spherocity index.

In some embodiments, the method can include operating a molecular graph generator to obtain molecular graph data and latent code data to construct a conformation of a molecule with a set of internal coordinates. Also, the molecular graph generator can convert the internal coordinates into Cartesian coordinates and perform at least one optimization to correct local distance geometry of at least one molecular substructure. The method may also include operating a conformation discriminator to distinguish between real conformations of a molecule from synthetic conformations of the molecule.

Additionally, the method can include operating an encoder to construct an irredundant latent space of latent data of input molecules. Also, the operation of the encoder can prevent mode collapse. The method can include operating a latent variables discriminator to map conformations into the latent space and to make the latent space similar to a normal prior distribution (e.g., conformations).

In some embodiments, the method can include determining a reconstruction loss between an original conformation of a molecule compared to a reconstructed conformation of the molecule. The reconstruction loss determination can be performed by adversarial analysis between the molecular graph generator against the confirmation discriminator and latent variables discriminator.

In some embodiments, the method can include constructing a first conformation having a rotation and translation invariant representation. Then, distances can be predicted between neighboring atoms of the first confirmation.

In some embodiments, the method can include considering a potential energy of a plurality of conformations. Then, physically plausible conformations can be selected based on the potential energy of each selected conformation. That is, lower potential energy conformations can be selected, and higher potential energy conformations can be discarded.

In some embodiments, a method can include modeling at least one provided conformation of the molecule with a biological target and determine whether or not the at least one provided conformation modulates the biological target. This can be by computer modeling with digital representations of the conformation and the biological target, such as by docking modeling, or by obtaining a physical version the molecule in the conformation and testing with a physical version of the biological target for biological activity (e.g., modulation).

In some embodiments, the method can include operating a graph convolution block to: update representations of nodes and edges of a molecule graph data; update node states; and/or update hidden states of edges.

In some embodiments, the method can include inputting condition data into the machine learning platform for use in generating conformations. In some aspects, the condition data is at least one conformation of the molecule.

In some embodiments, the method can include encoding discrete features of nodes and edge features with embedding layers. Accordingly, each edge feature can include a first graph subset that has a chemical bond type and bond stereochemistry. A sequence of graph convolution blocks can be applied to the discrete features to obtain an embedding of the molecular graph of the molecule.

In some embodiments, the method can include an encoder: obtaining a description of a conformation from molecular graph data of a molecule; and transforming the conformation with a sequence of graph convolution blocks to obtain node-wise latent codes. In some aspects, the latent codes are stochastic and sampled with reparameterization from a normal distribution parameterized by outputs of the encoder.

In some embodiments, the method can include a latent variables discriminator distinguishing generated latent codes of real conformations from noise and determining node-wise latent codes that are independent of each other and node-wise latent codes that follow the normal distribution.

In some embodiments, the method can include a conformation discriminator controlling quality of generated objects by assessing likelihood of conformations and determining quality of conformations based on potential energy estimations.

In some embodiments, the method can operate a conformation discriminator for passing molecular graph embeddings through a plurality of SchNet layers to obtain node representations and obtaining one aggregated value for the whole molecular conformation.

In some embodiments, the method can include determining an ability to synthesize a generated molecular conformation. In some aspects, the generated molecular conformation has at least one three dimensional restriction. The determination can be by determine the steps of the synthesis, and the ability to perform the steps. The difficulty of synthesis can also be ranked.

In some embodiments, one or more non-transitory computer readable media are provided for storing instructions that in response to being executed by one or more processors, cause a computer system to perform operations. The operations can include: obtaining molecule graph data for a molecule; inputting the molecule graph data into a machine learning platform; generating a plurality of conformations for the molecule with the machine learning platform, wherein the plurality conformations are specific to the molecule, each conformation having internal coordinates defining positions of atoms of the molecule; selecting at least one conformation for the molecule based on at least one parameter related to molecular conformations; and preparing a report that includes the selected at least one conformation for the molecule.

In some embodiments, a computer system can include: one or more processors; and one or more non-transitory computer readable media storing instructions that in response to being executed by the one or more processors, cause the computer system to perform operations. The operations can include: obtaining molecule graph data for a molecule; inputting the molecule graph data into a machine learning platform; generating a plurality of conformations for the molecule with the machine learning platform, wherein the plurality conformations are specific to molecule, each conformation having internal coordinates defining positions of atoms of the molecule; selecting at least one conformation for the molecule based on at least one parameter related to molecular conformations; and preparing a report that includes the selected at least one conformation for the molecule. The machine learning platform can include architecture of a molecular graph generator, conformation discriminator, stochastic encoder, and latent variables discriminator.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1A illustrates a system for a generative adversarial approach for conformational space modeling of molecules.

FIG. 1B illustrates modules that can be part of the system of FIG. 1A.

FIG. 1C shows architecture of a BORSCHT model.

FIG. 1D shows architecture of a COSMIC model.

FIG. 1E illustrates a generation protocol and molecular conformation space modeling with adversarial architecture.

FIG. 2 shows the samples from BORSCHT model trained on the GEOM-Drugs dataset.

FIG. 3 shows an overlay of 50 conformations from the ground-truth, proposed BORSCHT model and three baseline models for six molecular graphs from the GEOM-Drugs dataset.

FIG. 4 shows an overlay of 50 conformations from the ground-truth, proposed BORSCHT model and three baseline models for six molecular graphs from GEOM-QM9 dataset.

FIG. 5 shows the visualization of ground-truth and generated conformations by the COSMIC model and baselines.

FIG. 6A shows the RED values distributions on GEOM-DRUGS.

FIG. 6B shows the RED values distributions on GEOM-QM9 datasets.

FIG. 7 illustrates a generator network architecture.

FIG. 8 illustrates the encoder network architecture.

FIG. 9 illustrates the latent discriminator network architecture.

FIG. 10 illustrates the confirmations discriminator network architecture.

FIG. 11 shows the visualization of ground-truth and generated conformations by COSMIC model and baselines on GEOM-QM9 dataset.

FIG. 12 shows the individual samples from the COSMIC model trained on the GEOM-QM9 dataset.

FIGS. 13A-13B show the individual samples from the COSMIC model trained on the GEOM-Drugs dataset.

FIG. 14 shows an example computing device (e.g., a computer) that may be arranged in some embodiments to perform the methods (or portions thereof) described herein.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

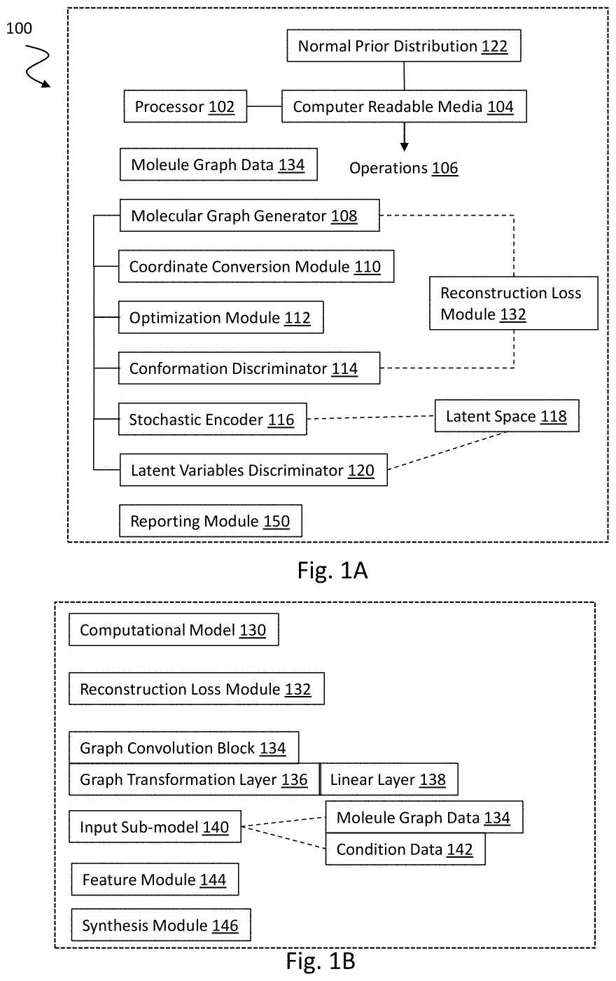

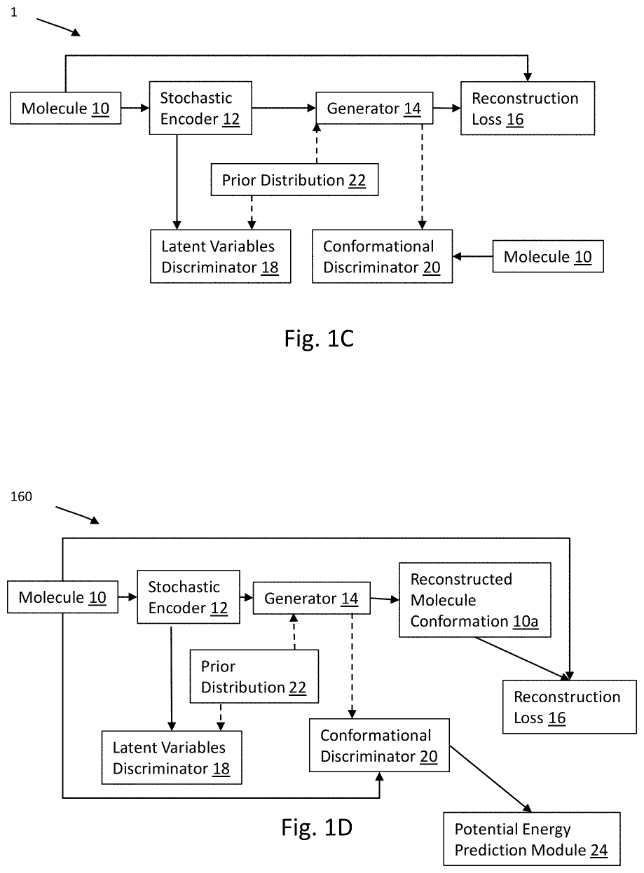

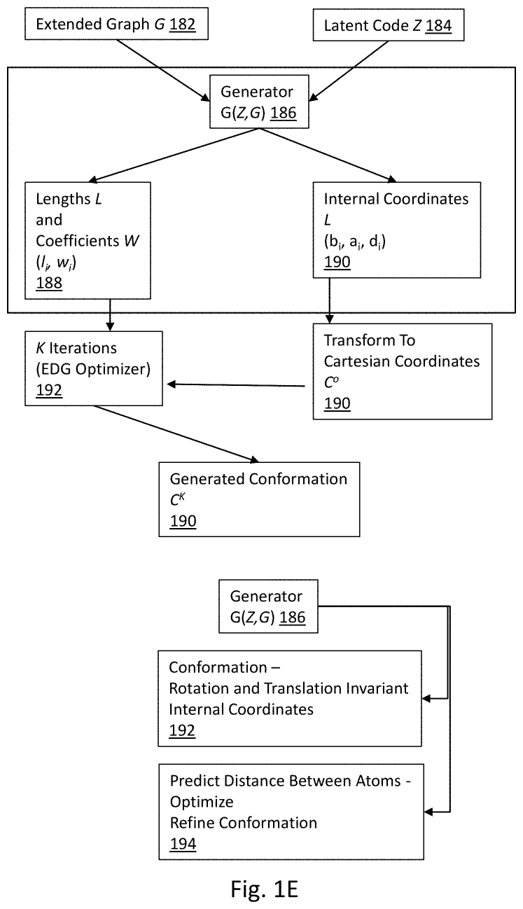

Recent neural-based approaches for molecular conformation generation show high diversity and target space coverage metrics but suffer from the lack of physical plausibility of generated structures. The present technology provides a novel adversarial generative framework to address this issue. The generator of this network produces a conformation in two stages, given a molecular graph and random noise. First, it constructs a conformation in a rotation and translation invariant representation (192, FIG. 1E)—internal coordinates of a conformation—an iterative representation, describing each atom by bond length and two angles relative to its predecessors. In the second step, the generator model predicts the distances between neighboring atoms and makes several optimization steps to refine the initial conformation (194, FIG. 1E). The generator predicts the distance geometry of local molecular structures and makes several optimization steps to refine the conformation. The proposed model considers conformation energy and produces physically plausible samples, outperforming previous models by energy metrics while achieving comparable results on space coverage and diversity metrics. The proposed model produces diverse low-energy conformations while showing geometric and coverage metrics comparable to previous models. The protocol can also introduce a simple yet meaningful conditional generation task and a corresponding benchmark—modeling of conformations with desired 3D descriptors for drug discovery problems like protein binding.

Generally, the present technology includes a conformation space modeling in internal coordinates (COSMIC) in a generative adversarial framework for roto-translation invariant conformation space modeling. The proposed approach benefits from combining internal coordinates and pair-wise distances iterative refinement. Additionally, the present technology includes a novel relative energy difference (RED) metric that exposes the physical plausibility of generated conformations by accounting for conformation energy. Also, the present technology provides a mechanism to introduce a novel conditional distribution learning task for generating conformations with provided 3D descriptors. The description herein provides extensive experiments on conformation distribution learning tasks for unconditional and conditional setups.

FIG. 1A illustrates a system 100 for a generative adversarial approach for conformational space modeling of molecules. The system 100 can include one or more processors 102; and one or more non-transitory computer readable media 104 storing instructions that in response to being executed by the one or more processors 102, cause the computer system 100 to perform operations 106 for a method of modeling molecule 3D conformations. The operations 106 can be performed as described by the modules of the system 100.

A molecular graph generator 108 is provided. The protocol can include operating the molecular graph generator 108 to obtain molecular graph data and latent code data in order to process the data and construct a conformation of a molecule with a set of internal coordinates. The internal coordinates define the relative positions of the atoms. The protocol can convert the internal coordinates into Cartesian coordinates with a coordinate conversion module 110. Then, there can be a performance of at least one optimization protocol with an optimization module 112 to correct local distance geometry of at least one molecular substructure.

A conformation discriminator 114 is provided. The protocol can include operating the conformation discriminator 114 to distinguish between real conformations of a molecule from synthetic conformations of the molecule.

A stochastic encoder 116 is provided. The protocol can include operating a stochastic encoder 116 to construct an irredundant latent space 118 of latent data of input molecules. Also, operation of the stochastic encoder 116 can be performed so as to prevent mode collapse.

A latent variables discriminator 120 is provided. The protocol can include operating the latent variables discriminator 120 to map conformations into the latent space 118. Also, the latent variables discriminator 120 can make the latent space 118 similar to a normal prior distribution 122.

Additionally, the system 100 can be operated to provide a computational model 130 having architecture of the molecular graph generator 108, conformation discriminator 114, stochastic encoder 116, and latent variables discriminator 120, as shown in FIG. 1B. The computational model 130 can be configured to determine a reconstruction loss, with a reconstruction loss module 132, between an original conformation of a molecule compared to a reconstructed conformation of the molecule. The reconstructed conformation is obtained from the computational model 130, with the system of FIG. 1A. For example, the reconstruction loss can be by an adversarial analysis between the molecular graph generator 108 against the confirmation discriminator 114 and latent variables discriminator 120 as per FIG. 1A. Also, the computational model 130 can be configured to obtain a prior distribution of graph data (e.g., normal prior distribution 122) and provide the prior distribution of graph data to the molecular graph generator 108 and the latent variables discriminator 120.

In some embodiments, the computational model 130 can include a graph convolution block 134 (e.g., module) that is configured to update representations of nodes and edges of a molecule graph data 134. Also, the graph convolution block 134 can include a graph transformation layer configured to update node state. Also, the graph convolution block 134 includes a linear layer 138 with residual connections configured to update hidden states of edges.

In some embodiments, the system 100 or computation model 130 can include one or more input sub-models 140 that are configured to obtain inputs from a molecular graph data 134 and a condition data 142. In some aspects, the condition data 142 can include conformation data, with each conformation having associated data thereof (e.g., 3D data).

The system 100 can also include a feature module 144 that is configured for performing an operation of encoding discrete features of nodes and edge features with embedding layers. In the feature module 144, each edge feature can have feature data including a first graph subset that has chemical bond type and bond stereochemistry. Also, the feature module 144 can be configured for applying a sequence of graph convolution blocks 134 to the discrete features to obtain an embedding of the molecular graph (e.g., molecule graph data 134) of the molecule. This can be done for each molecule that is input (e.g., training) or created by the molecular graph generator 108.

Additionally, the feature module 144 can be configured for encoding discrete features of nodes and edge features with embedding layers for each molecule. For example, each edge feature can include a first graph subset that has a chemical bond type and bond stereochemistry. This can define the 3D conformation of a molecule in space. Also, the feature module can apply a sequence of graph convolution blocks 134 to the discrete features to obtain an embedding of the molecular graph of the molecule.

In some embodiments, the molecular graph generator 108 is configured for generating information about molecules, such as the internal coordinates thereof, or other parameters. The operations of the system 100 can include operating a molecular graph generator 108 in two stages. The first stage in the molecular graph generator 108 can include generating the internal coordinates of a conformation of a molecule, which can be performed to a set of molecules, whether input or generated. The graph generator 108 also converts the internal coordinates into Cartesian coordinates for the molecule. In the second stage, the molecular graph generator 108 can predict lengths for each molecular graph bond of a molecule. Also, the molecular graph generator 108 can provide optimization rates for molecular graph bonds for one or more molecules. The molecular graph generator 108 can also refine distance geometry of local molecular structures of each molecule, such as atom placement, bond length, 3D spatial, and the like. Also, the molecular graph generator 108 can initialize with Cartesian coordinates from the first stage.

In some embodiments, the graph convolution block 134 can be a series of such blocks, which can be in a graph convolution block network. The graph convolution network can include a molecular graph embedding module, M-layer main body, and two 1-layer heads to predict generated internal coordinates of the conformation of the molecule or a plurality of conformation for each molecule.

In some embodiments, the optimization module 112 can perform optimization protocols for optimizing the atoms and bonds in a molecule for a 3D conformation. Accordingly, the operations performed by the system 100 include optimizing node positions to match the inter-atomic distances to the second, third, and fourth-order neighbors. Accordingly, the molecules can be optimized for conformation.

In some embodiments, the stochastic encoder 116 can be configured for obtaining latent data of the molecular conformations in the latent space 118. Accordingly, the operations can include the encoder 116 obtaining a description of a conformation from molecular graph data of a molecule. Then, the encoder 116 can transform the conformation of the molecule with a sequence of graph convolution blocks 134 to obtain node-wise latent codes for each molecule. In some aspects, the encoder 116 can be configured such that the latent codes are stochastic and sampled with reparameterization from the normal prior distribution 122 parameterized by outputs of the encoder 116.

In some embodiments, the latent variables discriminator 120 is configured for distinguishing between the latent codes of real conformations versus noise, which improves the output. As such, the operations include the latent variables discriminator 120 distinguishing generated latent codes of real conformations of a molecule from noise. The latent variables discriminator 120 can determine: node-wise latent codes that are independent of each other; and node-wise latent codes that follow the normal distribution.

In some embodiments, the conformation discriminator 114 can result in higher quality 3D conformations of each molecule. The operations can include the conformation discriminator 114 controlling the quality of generated molecule objects by: assessing likelihood of conformations; and determining quality of conformations based on potential energy estimations. Also, the operations include the conformation discriminator 114 passing molecular graph embeddings through a plurality of SchNet layers to obtain node representations; and obtaining one aggregated value for the whole molecular conformation.

The system 100 can also include a reporting module 150 configured for compiling and/or providing a report on one or more 3D conformations for each molecule. Accordingly, the operations can include the reporting module 150 reporting the coordinates of a generated conformation of a molecule. This can be done for each conformation. As such, a report can include one or more conformations, which may be ranked for the internal coordinates thereof, such as by energy. The operations can include the reporting module 150 providing the generated molecular conformation for the molecule. The report can include the generated molecular conformation for each conformation, which includes data on atom coordinate positions and/or bond data. This data defines the 3D conformation of the molecule. Also, the report of the data of the generated molecular conformation can be saved in a database of molecular conformations for the molecule (e.g., computer readable media 104, or a database on a memory device).

In some embodiments, the system 100 can include a synthesis module 146 that is configured to determine whether or not the molecule can be synthesized, and may provide a rating on the difficulty of synthesis. As such, molecules that are easier to synthesize can be prioritized. For example, a retrosynthesis protocol can be performed by the synthesis module 146. For example, WO 2012/229454 provides a protocol for assessing the retrosynthesis-related synthetic accessibility, which is incorporated by reference herein. Also, PCT/IB2021/061093 also teaches retrosynthesis techniques that are incorporated herein and can be used for determining molecules that can be synthesized, and optionally rank such synthesis schemes. This allows the system to determine the ability to synthesize a generated molecular conformation, wherein the generated molecular conformation has at least one three dimensional restriction.

In some embodiments, the computation model 130 can be trained with molecule data, which may include confirmation data for each molecule. As such, the operations may include performing a training of the computation model 130 with eth data. During the training, the model 130 can be improved by minimizing, for a training step, a reconstruction loss (e.g., reconstruction loss module 132) between an original conformation of a molecule compared to a reconstructed conformation of the molecule by adversarial analysis between the molecular graph generator 108 against the confirmation discriminator 114 and latent variables discriminator 120.

FIG. 1C shows architecture of a BORSCHT model 1 that can be used with the present invention. That is, the system can be configured with the elements of the BORSCHT model 1 and operate to provide the 3D conformation of the input molecule, for each input molecule. As shown, the molecule 10 is input into the stochastic encoder 12. The output of the stochastic encoder 12 can be provided to the generator 14 for generating the 3D conformations of the molecule 10. The stochastic encoder 12 also provides output to the latent variables discriminator 18 that makes a comparison with the prior distribution 22 as described herein. Also, the prior distribution 22 is used with the generator 14 for the protocol of generating the 3D conformations. The conformational discriminator 20 can receive output of the conformation from the generator 14 and make a comparison with the molecule 10, as described herein. Also, a reconstruction loss 16 can be determined for the molecule 3D conformation from the generator 14 compared to the input molecule 10.

FIG. 1D shows architecture of a COSMIC model 160, and the interaction therebetween. The COSMIC model 160 is similar to the BORSCHT model 1. Also, the reconstructed molecule conformation 10a can be obtained by the generator 14 in the BORSCHT model 1 even thought it is not shown. In FIG. 1D, the reconstructed molecule conformation 10a is compared for reconstruction loss 16 with the original input molecule 10. Additionally, there is a potential energy prediction module 24 that determines the predicted potential energy of the molecule conformation, which tells the energy of the molecular conformation. The potential energy for each conformation can be used for ranking or other uses. The potential energy can be used for accessing the plausibility of the generated 3D conformation for the molecule, and the most likely 3D conformation can be determined from the ranked potential energies of the different 3D conformations for the input molecule 10a.

FIG. 1E illustrates a generation protocol and molecular conformation space modeling with adversarial architecture. As shown, the Generator G takes an extended molecular graph G and latent codes Z to produce the lengths L and the coefficients W parameters for EDG and internal coordinates I. Next, the internal coordinates I are transformed into Cartesian Coordinates C° and used as a starting point in the EDG protocol. The optimization algorithm is used for K iterations to obtain conformation C^{K}.

In view of FIGS. 1A-1E, methods for a generative adversarial approach for conformational space modeling of molecules can be performed.

In some embodiments, a method for a generative adversarial approach for conformational space modeling of molecules is provided. The method can include inputting molecular data into a computing system configured with a model for adversarial approach for conformational space modeling of molecules. Then, the internal coordinates can be generated for input molecules or of generated molecules. The generated internal coordinates can contain representations that are iterated to describe a subsequently generated atom relative to a previously generated atom. Then, the generated conformation of the input molecules and/or the generated molecules can be determined. The conformation can include the internal coordinates of the molecule. The conformation of the molecule can be reported. Such a report may include the conformations of one or more molecules, and optionally a ranking thereof. The ranking may be by the reconstruction loss or the predicted potential energy.

In some embodiments, the method for conformational space modeling can include obtaining molecular graph data that includes a representation of the structural formula of a molecule in terms of graph theory. In such a structural formula, the nodes represent atoms and edges represent the corresponding chemical bonds. At least one molecular conformation for each molecular graph of the data can be obtained. As such, each molecular conformation can have a potential energy and a probability of being in the respective molecular conformation. The conformation space for each molecule can be obtained from a set of the molecular conformations, which are overlaid with respect to each other.

In some embodiments, the method for conformational space modeling can include obtaining each molecular conformation for each molecule in a set of Cartesian coordinates. That is, the coordinates can be converted to Cartesian coordinates. Then, each molecular conformation in a distance matrix D representation can be obtained. This can include pairwise Euclidean distances between atoms, such as a bond length. Each molecular confirmation can be obtained in an internal coordinates representation (e.g., which can be converted to Cartesian coordinates. The coordinate representation can include data for the bond length data, torsion angle data, and dihedral angle data to define a relative atom position of each subsequently generated atom relative to each previously generated atom for a molecule.

In some embodiments, the method for conformational space modeling can include steps for obtaining the internal coordinates of the internal coordinate representation. This can include constructing a spanning tree S of the molecular graph data and assigning a graph traversal order starting with a hanging node. The internal coordinates can be determined by unit direction and unit normal vectors with respect to indices of consecutive nodes in a graph transversal order.

In some embodiments, the method for conformational space modeling can include representing the molecular graph by nodes and edge feature sets and extending the molecular graph with auxiliary nodes and edges to make a proposed generative model. Then, virtual edges can be inserted between the second, third, and fourth neighboring nodes. Each node can be set to include a description of: atom type, charge, and chiral tag. Also, each edge feature can be set to include a first graph subset that has chemical bond type and bond stereochemistry. Also, the setting of each edge feature can include a second graph subset that has a spanning tree traversal process and having defining edge features to be in the spanning tree and information regarding whether a source node appears earlier in the spanning tree traversal process than a destination node. The method can also include optimizing node positions to match the inter-atomic distances to the second, third, and fourth-order neighbors.

In some embodiments, the method for conformational space modeling can include estimating the following conformation properties for each generated molecule: asphericity, eccentricity, inertial shape factor, two normalized principal moments ratios, three principal moments of inertia, gyration radius and sphericity index.

In some embodiments, the method for conformational space modeling can include operating a molecular graph generator to obtain molecular graph data and latent code data to construct a conformation of a molecule with a set of internal coordinates. Then, there can be a step to convert the internal coordinates into Cartesian coordinates. At least one optimization can be performed to correct local distance geometry of at least one molecular substructure (e.g., 3D conformation). A conformation discriminator can be used to distinguish between real conformations of a molecule from synthetic conformations of the molecule. A stochastic encoder can be used to construct an irredundant latent space of latent data of input molecules and prevent mode collapse. A latent variables discriminator to map conformations into the latent space and to make the latent space similar to a normal prior distribution.

In some embodiments, the model can be trained. As such, a method can include performing a training and minimizing, for a training step, a reconstruction loss between an original conformation of a molecule compared to a reconstructed conformation of the molecule by adversarial analysis between the molecular graph generator against the confirmation discriminator and latent variables discriminator. The training can be performed with the machine learning platform described herein. The determination of the reconstruction loss can be made between an original conformation of a molecule compared to a reconstructed conformation of the molecule by adversarial analysis between the molecular graph generator against the confirmation discriminator and latent variables discriminator.

In some embodiments, the method for conformational space modeling can include use of a prior distribution of graph data, such as for one or more 3D conformations for one or more molecules, such as the target molecule. A prior distribution of graph data can be provided to the molecular graph generator and the latent variables discriminator.

In some embodiments, the method for conformational space modeling can include using a graph convolution block that is configured to update representations of nodes and edges of a molecule graph data. Also, the graph convolution block can include a graph transformation layer configured to update node states. Additionally, the graph convolution block can includes a linear layer with residual connections configured to update hidden states of edges.

In some embodiments, condition data can be used for the protocols described herein. The method can include obtaining inputs from a molecular graph data and a condition, wherein the condition can be a conformation.

In some embodiments, the method for conformational space modeling can include a protocol for embedding of the molecular graph of the molecule. An encoding of discrete features of nodes and edge features can be obtained with embedding layers. Each edge feature can include a first graph subset that has chemical bond type and bond stereochemistry. A sequence of graph convolution blocks can be applied to the discrete features to obtain an embedding of the molecular graph of the molecule.

In some embodiments, the method for conformational space modeling can include operating a molecular graph generator in two stages. The first stage can include generating internal coordinates of a conformation, and converting the internal coordinates into Cartesian coordinates. The second stage can include predicting lengths and optimization rates for molecular graph bonds, and refining distance geometry of local molecular structures, wherein the predicting initializes with Cartesian coordinates from the first stage.

In some embodiments, the method for conformational space modeling can include predicting internal coordinates of a molecule. This can include operating a graph convolution network containing a molecular graph embedding module, M-layer main body, and two 1-layer heads to predict generated internal coordinates of the conformation.

In some embodiments, the method for conformational space modeling can include an encoder that obtains a description of a conformation from molecular graph data of a molecule, and transforms the conformation with a sequence of graph convolution blocks to obtain node-wise latent codes. The latent codes can be stochastic and sampled with reparameterization from normal distribution parameterized by outputs of the encoder.

In some embodiments, the method for conformational space modeling can include the latent variables discriminator distinguishing generated latent codes of real conformations from noise and determining node-wise latent codes being independent of each other and node-wise latent codes follow the normal distribution. The conformation discriminator can control the quality of generated objects by assessing likelihood of conformations (e.g., potential energy) and determining quality of conformations based on potential energy estimations. Also, the conformation discriminator can be used for passing molecular graph embeddings through a plurality of SchNet layers to obtain node representations and obtaining one aggregated value for the whole molecular conformation.

In some embodiments, the method for conformational space modeling can determine whether or not a molecule with a specific conformation can be synthesized or the level of difficulty of such synthesis. This can include a module determining the ability to synthesize the generated molecular conformation, wherein the generated molecular conformation has at least one three dimensional restriction.

In some embodiments, the method for conformational space modeling can provide the generated one or more molecular conformations for the molecule. This can then be used for synthesizing the molecule in order to obtain the generated 3D conformation. Also, the conformation can be used in modeling studies with the molecule and a biological target. As such, a report can be generated with the generated molecular confirmation or confirmation space and the data thereof. The generated molecular conformation can include data on atom coordinate positions. The generated molecular conformation can be saved in a database of molecular conformations for the molecule. The coordinates of a generated conformation of a molecule can be included in the report, which can be used to compare with a synthesized molecule for obtaining the 3D conformation.

A molecular graph G is a representation of the structural formula of a molecule in terms of graph theory, where nodes represent atoms and edges represent the corresponding chemical bonds. Molecular conformation C of a molecular graph G is a 3D realization of a molecule existing in the real world. Each molecule can be found in nature in an infinite set of conformations with probabilities proportional to the potential energy U(C,G) and forms a conformation space p(C|G)∝exp(−U(C,G)). The conformations with lower potential energy are more plausible.

In real-world problems, the environment imposes external restrictions R on the molecular conformation. In this case, interaction energy UR between the molecule and the environment affects the conformation space of the molecular graph G: p(C|G, R)∝exp(−U(C,G)+U_{R}(C,G,R)). Neural conformation space modeling for both restricted and unrestricted cases is described herein.

A way to represent and store molecular conformation C is a set of Cartesian coordinates Cᵢ=(xᵢ, yᵢ, zᵢ) of atoms. Although this representation is intuitive and easy to work with, it suffers from a lack of translation, rotation, and reflection invariances. Another representation is a distance matrix D that stores pairwise Euclidean distances between atoms Dᵢⱼ=∥Cᵢ−Cⱼ∥₂. It provides translation, rotation, and reflection invariances in contrast to Cartesian representation. However, this format is overparameterized and contains implicit dependencies between the matrix elements: the distances between every triplet of points should satisfy the triangle inequality, which making it challenging to learn the distribution over the conformations by modeling distance matrices.

Finally, internal coordinates representation I={(bᵢ, aᵢ, dᵢ)} describes molecular conformation iteratively atom by atom. Bond length bᵢ ∈R⁺, torsion angle (bond angle) aᵢ ∈[0, π), and dihedral angle dᵢ ∈[0, 2π) specify a relative atom position to the predecessors. To obtain internal coordinates, one needs to construct a spanning tree S of the molecular graph G and assign a graph traversal order starting with a hanging node. Let l, k, j, i be the indices of consecutive nodes in the graph traversal process. Then, in terms of unit direction

$$\hat{{u}_{ij}} = \frac{{C}_{i} - {C}_{j}}{‖ {C}_{i} - {C}_{j} ‖}$$

and unit normal vectors

$$\hat{{u}_{ijk}} = \frac{\hat{{u}_{jk}} \times \hat{{u}_{ij}}}{‖ \hat{{u}_{jk}} \times \hat{{u}_{ij}} ‖}$$

internal coordinates are:

$${b}_{i} = ‖ {C}_{i} - {C}_{j} ‖ , {a}_{i} = arccos ( - \hat{{u}_{ij}} \cdot \hat{{u}_{jk}} ) , {d}_{i} = sign ( \hat{{u}_{kl}} \cdot \hat{{u}_{ijk}} ) arccos ( \hat{{u}_{jkl}} \cdot \hat{{u}_{ijk}} ) \qquad ( 1 )$$

The first three nodes of a graph traversal have an insufficient number of predecessors. Therefore, its missing coordinates receive zero values. Internal coordinates are translation and rotation invariant but not reflection invariant. The enantiomers (mirrored conformations) differ only in the sign of dihedral angle and can be easily computed from the original conformation as Iᵣ={(bᵢ, aᵢ, −dᵢ)}. All three representations can be used in different parts of the generative model in a way that emphasizes its strengths and neglects its weaknesses.

The protocol represents a molecular graph G by nodes and edges feature sets (V,E). The protocol extends the molecular graph with auxiliary nodes and edges to make the proposed generative model more flexible. The protocol adds virtual edges between the second, third, and fourth neighboring nodes. In special cases, the protocol adds a fictitious node when the molecular spanning tree does not have a hanging chain of three heavy atoms to start an internal coordinates computation. An example of this case is the isobutane molecule HC(CH₃)₃.

The protocol extends the molecular graph with auxiliary nodes and edges to make the proposed generative model more flexible. Similar to prior protocols (Xu et al. [2021a]; Simm, et al. [2020]), the protocol can add virtual edges between the second and third neighboring nodes. The protocols adds a fictitious node when the spanning tree of the molecular graph does not have a hanging chain of at least two heavy atoms to start an internal coordinates computation.

Set V contains nodes description: an atom type, charge, and a chiral tag for each node. The protocol assigns a special value to the fictitious node attributes. Set E comprises two subsets Eₘ, Eₛ of directed edge features. Since the original molecular graph and auxiliary edges are undirected, each edge in E has a duplicate with swapped source and destination nodes.

Features group Eₘ contains a chemical bond type and bond stereometry tag for each edge. The protocol assigns a fictitious stereometry tag and use graph distance as a bond type for virtual edges. Features group Eₛ describe the spanning tree traversal process to make internal coordinates modeling easier. Group Eₛ stores boolean features that describe whether an edge is in the spanning tree and whether a source node appears earlier in the spanning tree traversal process than a destination node.

In some embodiments, the protocol represents a molecular graph=(V, E) by feature sets of nodes V and edges E. In particular, the set V contains an atom description: atom type, charge, and a desired chiral tag for each node. Edge features E contain chemical bond type and bond stereochemistry tag for each edge. Each edge in E has a duplicate with swapped source and end nodes. The protocol assigns unique values to the attributes of fictitious nodes and edges. Additionally, the protocol includes the spanning tree traversal process description into E to make internal coordinates easier to model.

In some embodiments, the protocol uses conformation descriptors from the Descriptors3D module of RDKit (Landrum [2016]) as an example of external restrictions. The protocol estimates the following conformation properties: asphericity, eccentricity, inertial shape factor, two normalized principal moments ratios, three principal moments of inertia, gyration radius and spherocity index. The description of these 3D properties can be found in the supplementary material.

BORSCHT

In some embodiments, the BORSCHT model can be used for molecular conformation generation. The BORSCHT model 1 can include an architecture that has four parts. The first part (1) is the generator G_{ψ}(Z,G) 14 that takes molecular graph G and latent code Z as input and constructs a conformation C as a set of internal coordinates, converts them into Cartesian coordinates and makes several coordinates optimization steps to correct the local distance geometry of molecular substructures for a conformation reconstruction loss 16 (R(C, Ĉ). (2) The conformation discriminator D_{ψ}^{conf} (C, G) 20 is based on the SchNet architecture, and it tries to distinguish the real conformations from synthetic ones. To construct an irredundant latent space and prevent the model from mode collapse, the protocol also introduces (3) a stochastic encoder E_{θ}(C,G) 12 and (4) a latent variables discriminator D_{ψ}^{lat} (Z, G) 18 to map conformations onto the latent space and make this latent space similar to the normal prior distribution 22. FIG. 1 shows architecture of the BORSCHT model.

Generally, the BORSCHT model 1 is a combination of the Wasserstein Generative Adversarial Network (WGAN) (Arjovsky et al. [2017]) with Gradient Penalty (Gulrajani et al. [2017]) and the Adversarial Autoencoder (AAE) (Makhzani et al. [2015]). On each training step, the generator and the encoder play an adversarial game against two discriminators while trying to minimize the reconstruction loss R between original and reconstructed conformations:

$${L}_{G , E} = {E}_{C , G \sim {P}_{x}} [ {E}_{Z \sim {E}_{θ ( C , G )}} [ R ( C , {G}_{ω} ( \hat{Z} , G ) ) + {λ}_{l} log {D}_{ϕ}^{l a t} ( \hat{Z} , G ) ] + {λ}_{c} {E}_{Z \sim {P}_{z}} {D}_{ψ} ( {G}_{ω} ( Z , G ) , G ) ] \to {min}_{ω , θ} \qquad ( 2 )$$

$${L}_{{D}^{conf}} = {E}_{C , G \sim {P}_{x}} {[}_{{E}_{Z \sim {P}_{x}}} {D}_{ψ}^{conf} ( {G}_{ω} ( Z , G ) , G ) - {D}_{ψ}^{conf} ( C , G ) - {λ}_{GP} {{E}_{\overline{C} \sim {P}_{C}} ( {‖ {∇}_{\overline{C}} {D}_{ψ}^{conf} ‖}_{2} - 1 )}^{2} ] \to {max}_{ψ} \qquad ( 3 )$$

$${L}_{{D}^{lat}} = {E}_{C , G \sim {P}_{x}} [ {E}_{Z \sim {E}_{θ ( C , G )}} log {D}_{ψ}^{l a t} ( \hat{Z} , G ) + {E}_{Z \sim {P}_{z}} log ( 1 - {D}_{ϕ}^{l a t} ( Z , G ) ) ] \to {max}_{ϕ} \qquad ( 4 )$$

Here, P_{X}={(C,G)} is a data distribution, P_{Z}=N(0, l) is a prior distribution, P_{C} is a convex set, containing interpolation points between real and generated conformations, and coefficients λ_{GP}, λ_{c}, λₗ are the hyperparameters of the optimization problem.

In some embodiments, a graph convolution block is used. The basic building layer of the model is a graph convolution block. The protocol utilizes it to update the representations of nodes and edges. The block contains the Graph Transformer Layer (GTL) (Shi et al. [2020]) to update node states hₙ, and linear layer with residual connections to update hidden states of edges hₑ.

The protocol lets h[i] denote the operation of taking elements of an array h by the sequence of indices i, cat( . . . ) denote tensors concatenating along the last dimension, and arrays s and f denote the starting and final nodes of edges. Then graph convolutional block (GCB) can be introduced in the following way:

hₙ^{l+1}=LeakyReLU(GTL^{l+1}(hₙ^{l},hₑ^{l})) (5)

hₑ^{l+1}=LeakyReLU(hₑ^{l}+Linear(cat(hₑ^{l},hₙ^{l+1}[s],hₙ^{l+1}[f]))) (6)

In some embodiments, the molecular graph can be embedded. Similar to the conditional modification of GAN (Goodfellow et al. [2014]), all submodels of BORSCHT take a molecular graph G, the condition, as one of the inputs. The protocol encodes discrete features of nodes V and edges Eₘ with embedding layers and apply a sequence of L graph convolution blocks to obtain the embedding Emb(G) of the molecular graph:

Emb(G)=GCB_{emb}^{L}∘ . . . ∘GCB_{emb}¹(Emb(V),Emb(Eₘ)) (7)

Note that sub-models have independent graph embedding parts and do not share the weights among each other.

In some embodiments, the generator can be configured as described herein. A core part of the proposed model is the generator. The conformation generation proceeds in two stages. First, the model produces the internal coordinates of a conformation and converts them into Cartesian coordinates. On the second stage, the model predicts the lengths and optimization rates for molecular graph bonds, and makes several optimization steps to refine the distance geometry of local molecular structures, starting with the Cartesian coordinates given from the first generation step.

Formally, given a set of node-wise latent variables Z and a description of a molecular graph G, the protocol runs a graph convolutional network, containing molecular graph embedding part, M-layer main body, and two 1-layer heads to predict internal coordinates Î of the conformation and set of bond lengths and optimization rates \hat{D}:

(h_{emb}ⁿ,h_{emb}^{e})=Emb_{gen}(G) (8)

(hᵢₙₚⁿ,hᵢₙₚ^{e})=(h_{emb}ⁿ+Linear(Z),h_{emb}^{e}+Emb(Eₛ)) (9)

(hₒᵤₜⁿ,hₒᵤₜ^{e})=GCB_{gen}^{M}∘ . . . ∘GCB_{gen}¹(hᵢₙₚⁿ,hᵢₙₚ^{e}) (10)

\hat{I}=MLP(GCB_{ic}(hₒᵤₜⁿ,hₒᵤₜ^{e})) (11)

\hat{D}=MLP(GCB_{dg}(hₒᵤₜⁿ,hₒᵤₜ^{e})) (12)

The protocol uses an iterative formula, the inverse of equation 1 to convert the internal coordinates Î={\hat{b}ₗ, âₗ, \hat{d}ₗ} to Cartesian coordinates Ĉ⁰={\hat{x}ₗ, ŷₗ, \hat{z}ₗ}:

Ĉₗ⁰=Ĉⱼ⁰+bᵢ(cos(π−aᵢ)ûⱼₖ+sin(π−aᵢ)cos(dᵢ)[(\hat{n})ⱼₖₗx(û)ⱼₖ]+sin(π−aᵢ)sin(dᵢ)(\hat{n})ⱼₖₗ) (13)

Then, given a set \hat{D}{(\hat{l}ₗ, \hat{t}ₗ)} of predicted bond lengths \hat{l} and edge coefficients \hat{t}, The protocol makes K iteration of distance geometry optimization process, starting with Ĉ₀ coordinates:

$$L = {\sum }_{i} \frac{\hat{{t}_{l}}}{2} {( {‖ \hat{C} [ s ] - \hat{C} [ f ] ‖}_{2} - {\hat{d}}_{l} )}^{2} \to {min}_{C} \qquad ( 14 )$$

$$∀ k = 1 , … , K : {\hat{C}}^{k} = {\hat{C}}^{k - 1} - \frac{δ L}{δ \hat{C}} \vert {\hat{C}}^{k - 1} \qquad ( 15 )$$

In some aspects, the optimization problem contains terms for all edges, including the virtual ones. In other words, the protocol optimizes node positions to match the inter-atomic distances to the second, third, and fourth-order neighbors. The final coordinates C represent the generated conformation.

The reconstruction loss R(Ĉ, C) between reconstructed and original conformations can be computed in terms of the original internal coordinates I={(bᵢ, aᵢ, dᵢ)}, the predicted internal coordinates Î={\hat{b}ₗ, âₗ, \hat{d}ₗ}, the original distance matrix D, the distance matrix \hat{D} of reconstructed conformation and the matrix T of the shortest path lengths between nodes of the molecular graph:

$$R ( \hat{C} , C ) = \frac{2}{n ( n - 1 )} {\sum }_{i < j} \frac{D [ i , j ] - \hat{D} [ i , j ]}{T [ i , j ]} + {λ}_{c o s} {min}_{s i g n ∈ ∖ \{ - 1 , 1 ∖ \}} {\sum }_{i} [ cos ( {d}_{i} - {\hat{d}}_{l} ) + cos ( {d}_{i} - sign \cdot {\hat{d}}_{l} ) ] \qquad ( 16 )$$

The conformation encoder E_{θ}(C,G) takes the description of a conformation and its molecular graph as inputs and transforms them with a sequence of graph convolution blocks to the node-wise latent codes Z={zᵢ}. The latent codes obtained from the encoder are stochastic and sampled with reparameterization trick from normal distribution parameterized by outputs of the encoder. The size of the node latent code zᵢ is fixed, however, the total size grows proportionally to the number of nodes in the extended graph. To construct irredundant latent space, do not give the encoder direct access to the internal coordinates. Instead, use graph traversal and spanning tree features Eₛ, along with the edge lengths L.

The latent code discriminator D_{ψ}^{lat}(Z, G) tries to distinguish latent codes of real conformations from the Gaussian noise. Since Z={zᵢ} is structured and contains separate latent descriptions for each node, the discriminator should be able to check two statements: node-wise latent codes zᵢ are independent between each other (1) and follow the normal distribution (2). The paradigm of graph neural networks allows for seamless implementation of this type of discriminator.

Similar to the generator, the discriminator computes the embeddings of molecular graph G and edge features, linearly adds the transformed latent codes Z to the node hidden states and applies several graph convolution blocks to compute the final hidden state of each node. Thereafter, it computes mean and max pooling to aggregate a fixed-size representation and apply multi-layer perceptron (MLP) to obtain output values.

The conformation discriminator controls the quality of generated objects. The potential energy allows for the assessment of the likelihood of conformations and determines their quality. For the discriminator architecture, the protocol uses the SchNet architecture which is widely used in end-to-end conformation energy estimation problems.

The protocol passes the molecular graph embeddings through several SchNet layers to obtain node representations. Then, as in the latent discriminator, perform pooling and apply an MLP to obtain one aggregated value for the whole molecular conformation.

COSMIC

In some embodiments, the methods and models can be based on the COSMIC (COnformation Space Modeling in Internal Coordinates) framework. COSMIC combines two adversarial models, the Wasserstein Generative Adversarial Network with Gradient Penalty (WGAN-GP) and the Adversarial Autoencoder (AAE), which share the generator/decoder part. These two models complement each other—AAE provides an expressive latent space and diverse samples, while WGAN-GP controls the physical plausibility of produced samples.

In some embodiments, the conformation space can be obtained as having a molecular graph [symbol] that represents a molecular structure. In terms of graph theory, nodes represent atoms, and edges represent chemical bonds. The molecular conformation [symbol] of graph [symbol] is a 3D structure of a molecule. Each molecule can have an infinite set of conformations, where all possible conformations form a conformation space p( [symbol] | [symbol] )∝ exp(−U( [symbol] , [symbol] )) and have a distribution that depends on the potential energy U( [symbol] , [symbol] ).

In some embodiments, the conformation representation can be obtained. In some aspects, a way to represent molecular conformation C is to store Cartesian coordinates Cᵢ=(xᵢ, yᵢ, zᵢ) of atoms. Although this representation is intuitive and easy to work with, it lacks the invariance in translation, rotation, and reflection (E(3) symmetry group).

Another representation is a distance matrix D that specifies pairwise Euclidean distances between atoms Dᵢⱼ=∥Cᵢ−Cⱼ∥₂. It provides translation, rotation, and reflection invariances in contrast to Cartesian representation. However, this format is overparameterized and contains implicit dependencies between the matrix elements: the distances between every triplet of points should satisfy the triangle inequality, which making it challenging to learn the distribution over the conformations by modeling distance matrices.

Finally, internal coordinates representation I={(bᵢ, aᵢ, −dᵢ)} describes molecular conformation iteratively atom by atom in order of some traversal of the molecular graph [symbol] . Bond length bᵢ ∈ [symbol] ⁺, bond angle αᵢ ∈[0, π), and dihedral angle dᵢ ∈[0, 2π) specify the relative atom position to its predecessors. Internal coordinates are translation and rotation invariant but not reflection invariant. However, internal coordinates can easily model the enantiomers (mirrored conformations) that differ only in the sign of dihedral angles, i.e., Iᵣ={(bᵢ, aᵢ, −dᵢ)}.

In some embodiments, molecular graph features are used. The protocol can extend the molecular graph with auxiliary nodes and edges to make the proposed generative model more flexible. Similar to prior methods (Xu et al. [2021a]; Simm et al. [2020]), the protocol adds virtual edges between the second and third neighboring nodes. The protocol also adds a fictitious node when the spanning tree of the molecular graph does not have a hanging chain of at least two heavy atoms to start an internal coordinates computation.

The protocol represents a molecular graph [symbol] =(V, E) by feature sets of nodes V and edges E. In particular, the set V contains an atom description: atom type, charge, and a desired chiral tag for each node. Edge features E contain chemical bond type and bond stereochemistry tag for each edge. Each edge in E has a duplicate with swapped source and end nodes. The protocol assigns unique values to the attributes of fictitious nodes and edges. Additionally, the protocol includes the spanning tree traversal process description into E to make internal coordinates easier to model.

Generative Adversarial Networks (GAN) (Goodfellow et al. [2014]) include a family of generative models that learn the data distribution p_{d} by solving a min-max game between two neural networks—a generator G(z) and a discriminator D(x). The procedure reaches equilibrium when the generator can take a random noise z from prior p(z) and produce objects the optimal discriminator cannot distinguish from the real ones.

One of the most popular architectures in the GAN family is Wasserstein Generative Adversarial Network (WGAN) (Arjovsky et al. [2017]). It utilizes the minimization of the Wasserstein distance between real and generated distributions. (Gulrajani et al. [2017]) proposed to impose the discriminator's Lipschitz continuity (Zhou et al. [2019]) and minimize the gradient penalty (GP) at point \hat{x}, which is sampled uniformly between a pair of generated and real objects. The training objective of WGAN-GP is as follows:

$${min}_{G} {max}_{D} {𝔼}_{x \sim {p}_{d} , z \sim p ( z )} \underbrace{[ D ( x ) - D ( G ( z ) ) - {λ}_{GP} {ℒ}_{GP} ]}_{{ℒ}_{WGAN}} \qquad ( A )$$

$${{ℒ}_{G P} = {{𝔼}_{λ \sim U [ 0 , 1 ]} ( {‖ {∇}_{\hat{x}} D ( \hat{x} ) ‖}_{2} - 1 )}^{2} \vert}_{\hat{x} = λ x + ( 1 - λ ) G ( z )}$$

Another popular generative model that shares the benefits of the adversarial approach is Adversarial Auto Encoder (AAE) (Makhzani et al. [2015]). The encoder E(x) and decoder G(z) networks collaborate to learn an expressive latent representation z. Moreover, the latent code z should contain all necessary information about the object x to reconstruct it with the smallest possible error R. The latent discriminator D (z) tries to make the distribution of latent representations indistinguishable from the prior distribution p(z). The AAE training objective is as follows:

$${min}_{E , G} {max}_{D} \underbrace{{𝔼}_{x \sim {p}_{d}} {ℒ}_{data} + {𝔼}_{z \sim p ( z )} log ( 1 - D ( z ) )}_{{ℒ}_{AAE}} \\ {ℒ}_{data} = {𝔼}_{z \sim E ( x )} R ( x , G ( z ) ) + log D ( z ) \qquad ( B )$$

In total, COSMIC (FIG. 1D) includes four parts. In the first part, (1) the generator G( [symbol] , [symbol] ) takes a molecular graph [symbol] and a set of latent vectors for each node [symbol] ={zᵢ}ᵢ₌₁^{|V|} as input and constructs the conformation Ĉ as the sequence of internal coordinates. Further, the protocol transforms internal coordinates into the Cartesian format, and the Euclidean Distance Geometry (EDG) algorithm performs several optimization steps to correct the distances between the neighboring atoms. In the second part, (2) the conformation discriminator D^{conf}((C, [symbol] ) aims to distinguish the real conformations from generated and predict their energy difference. The protocol uses the following two modules to construct a convenient latent space and prevent mode collapse: (3) the encoder E(C, [symbol] ) maps conformations into the latent space, and (4) the latent variables discriminator D^{lat}( [symbol] , [symbol] ) makes the latent space similar to the prior distribution.

In some embodiments, the generator can be configured and operated as described herein, which is the generator G( [symbol] , [symbol] ). The conformation generation proceeds in stages, shown in FIG. 1E First, the graph neural network takes a molecular graph [symbol] and noise [symbol] to produce the internal coordinates Î of a conformation and predicts the parameters of the EDG problem—bond lengths [symbol] and bond-wise loss function weights Ŵ. In the second stage, the generator iteratively converts the internal coordinates Î={(\hat{b}ᵢ, âᵢ, \hat{d}ᵢ)} to the Cartesian representation Ĉ⁰. Let l, k, j, i be the indices of consecutive nodes in the graph traversal process. Then, the protocol computes the Cartesian coordinates in terms of unit direction

$${\hat{u}}_{ij} = \frac{{\hat{C}}_{i} - {\hat{C}}_{j}}{‖ {\hat{C}}_{i} - {\hat{C}}_{j} ‖}$$

and unit normal vectors

$${\hat{n}}_{ijk} - \frac{{\hat{u}}_{jk} \times {\hat{u}}_{ij}}{‖ {\hat{u}}_{jk} \times {\hat{u}}_{ij} ‖} :$$

Ĉᵢ⁰=Ĉⱼ⁰+\hat{b}ᵢ(−cos(âᵢ)ûⱼₖ+sin(âᵢ)sin(\hat{d}ᵢ)\hat{n}ⱼₖₗ+sin(âᵢ)cos(\hat{d}ᵢ)(\hat{n}ⱼₖₗ×ûⱼₖ)) (C)

After computing the coordinates Ĉ⁰, the protocol solves the EDG problem on bonds, where a starting node sᵢ and an ending node fᵢ define each bond. This forces bond lengths to match the predicted distances [symbol] ={Îᵢ}ᵢ₌₁^{|E|}. The optimization problem contains terms for all edges, including the virtual ones, i.e., between the first, second, and third-order neighbors. Also, the objective includes generator predicted coefficients Ŵ={Ŵᵢ}ᵢ₌₁^{|E|}, Ŵᵢ ∈[0, 1] that show the generator's confidence in the predicted bond length Îᵢ.

$${ℒ}_{EDG} = {\sum }_{1 \leq i \leq \vert E \vert} {{\hat{w}}_{i} ( {‖ {\hat{C}}_{{s}_{i}} - {\hat{C}}_{{f}_{i}} ‖}_{2} - {\hat{I}}_{i} )}^{2} {\hat{C}}^{k} = {\hat{C}}^{k - 1} - {∇}_{\hat{C}} {ℒ}_{EDG} ( {\hat{C}}^{k - 1} ) \qquad ( D )$$

The protocol runs the gradient descent algorithm for K steps to optimize the EDG objective L_{EDG}. The does not introduce an additional parameter for optimization step size since the generator can control it by alternating the coefficients Ŵ. The final coordinates Ĉ_{K} represent the generated conformation. While training, the protocol propagates gradient from reconstruction loss R through the steps of the optimization process in Eq. D to train the generator network.

In some embodiments, the reconstruction loss can be determined. A component of the proposed framework is the reconstruction loss R(Ĉ, C) between reconstructed and original conformations. It contains two parts. The first component controls the quality of an EDG problem solution. It takes ground-truth C and reconstructed coordinates Ĉ^{K} of n nodes of the molecular graph, and computes the absolute difference between D and D distance matrices. To encourage the consistency of local structures, the protocol divides the element-wise differences |D[i,j]−\hat{D}[i,j]| by lengths of the shortest paths T[i,j] in terms of edge hops. For this loss, the close neighbors are more important than the distant ones.

$${R}_{D} ( {\hat{C}}^{k} , C ) = \frac{2}{\vert V \vert ( \vert V \vert - 1 )} {\sum }_{1 \leq i < j \leq \vert V \vert} \frac{\vert D [ i , j ] - \hat{D} [ i , j ] \vert}{T [ i , j ]} \qquad ( E )$$

The second term controls the quality of internal coordinates Î={(\hat{b}ᵢ, âᵢ, \hat{d}ᵢ)} used to parametrize the starting conformation in the EDG problem. It contains the MSE loss on the predicted bond length and cosine losses on the predicted bond and dihedral angles. Since internal coordinates are not reflection-invariant, iterate over enantiomers, e.g., the conformations that differ from each other by the sign of a dihedral angle.

$${R}_{I} ( \hat{I} , I ) = \frac{1}{\vert V \vert} {min}_{s ∈ \{ - 1 , 1 \}} {\sum }_{1 \leq i \leq \vert V \vert} [ {( {b}_{i} - {\hat{b}}_{i} )}^{2} - cos ( {a}_{i} - {\hat{a}}_{i} ) - cos ( {d}_{i} - s \cdot {\hat{d}}_{i} ) ] \qquad ( F )$$

The protocol linearly combines these objectives to get the final reconstruction loss. The proposed reconstruction loss and, hence, the generator is translation, rotation, and reflection invariant.

R(Ĉ,C)=λ_{I}R_{I}(Î,I)+λ_{D}R_{D}(Ĉ^{k},C) (G)

In some embodiments, the confirmation encoder can be configured and operated as described herein. The encoder E(C, [symbol] ) takes the description of a conformation and its graph as inputs and transforms them by a sequence of graph convolutions into the node-wise latent codes [symbol] ={zᵢ}ᵢ₌₁^{|V|}. Although the node's latent code zᵢ size is constant, the total size grows proportionally to the number of nodes in the extended graph. The protocol does not give the encoder direct access to the internal coordinates to construct an expressive latent space; instead, the protocol uses edge lengths D.

In some embodiments, the conformation and latent discriminator can be configured and operated as described herein. The conformation discriminator D^{conf}(C, [symbol] ) controls the quality of generated objects. The protocol adopts the SchNet architecture for the discriminator, as it succeeded in conformation energy estimation problems. Graph convolution layers produce node embeddings, which are passed further through SchNet interaction layers along with inter-atomic distances. The resulting node representations are the input for two heads of the conformation discriminator.

The first output D^{conf}(C, [symbol] ) is for the WGAN-GP discriminator, while the second Ū(C, [symbol] ) predicts the energy of the conformation. To make the computation of gradient penalty in Eq. A more stable, the protocol aligns real C and generated Ĉ conformations. For the second head, the protocol uses an external function U(C, [symbol] ) to compute energies. The protocol trains Ū(C, [symbol] ) to predict the energy difference Δ_{U}=U(Ĉ, [symbol] )−U(C, [symbol] ) between the real and generated conformations:

[symbol]

_{U}=|(Ū(Ĉ, [symbol] )−Ū(C, [symbol] ))−Δ_{U}| (H)

The protocol can chose the MMFF94s algorithm of RDKit (Landrum [2016]) for U(C, [symbol] ) implementation as a trade-off between computation time and accuracy. However, such does not limit the methodology with this choice, and one can easily extend it to other algorithms that estimate energy more accurately.

Finally, the other latent code discriminator D^{lat}( [symbol] , [symbol] ) consists of several graph convolutions that take a molecular graph g and latent code [symbol] and distinguish latent codes of real conformations from the Gaussian noise.

In some embodiments, a training objective can be used. The optimized objective of the COSMIC is a linear combination of WGAN-GP, AAE, and energy prediction objectives in Eqs. A, B, H. The generator and the encoder play an adversarial game against two discriminators and minimize the reconstruction loss between original and reconstructed conformations.

$${min}_{E , G} {max}_{{D}^{conf} , {D}^{lat}} {ℒ}_{AAE} + {λ}_{WGAN} {ℒ}_{WGAN} - {λ}_{U} {ℒ}_{U} \qquad ( I )$$

The proposed COSMIC framework is roto-translation and reflection invariant. To ensure this property, the protocol (1) parameterizes initial conformation in internal coordinates, (2) refine this conformation iteratively, operating on interatomic distances only, and (3) use roto-translation and reflection invariant training objective and model subparts.

An example is provided for an extensive ablation study to experimentally evaluate the importance of distinct parts of the proposed loss and architecture.

In this work, a novel neural network architecture (e.g., platform) is proposed for conditional molecular conformational space modeling with internal coordinates and introduce an energy-based metric for conformation evaluation. Conducted experiments show that the approach outperforms previous state-of-the-art methods on the task of modeling conformational space with given external restrictions in the form of 3D descriptors.

The selected object (e.g., molecule with 3D conformation) is then provided to the object synthesizer (e.g., molecule synthesizer), where the selected object (e.g., selected molecule) is then synthesized. The synthesized object (e.g., molecule 3D conformation) is then provided to the object validator (e.g., molecule validator, which tests the object to see if it satisfies the condition of the 3D conformation. For example, a synthesized object that is a molecule can be tested with mass spectroscopy, NMR, x-ray diffraction, and other techniques to determine the 3D confirmation of the molecule. Other validation techniques in order to validate that the synthesized molecule satisfies the 3D conformation condition.

In some embodiments, the methods may include: obtaining a physical object for the selected molecule 3D conformation; and testing the physical molecule with the condition of the 3D conformation to see if the 3D conformation has been obtained. Also, in any method the obtaining of the physical molecule in the 3D conformation can include at least one of synthesizing, purchasing, extracting, refining, deriving, or otherwise obtaining the physical 3D conformation of the molecule. The methods may include testing the physical 3D conformation in a cell culture to assess bioactivity. The methods may also include assaying the physical 3D conformation by genotyping, transcriptome-typing, 3-D mapping, ligand-receptor docking, before and after perturbations, initial state analysis, final state analysis, or combinations thereof. Preparing the physical 3D confirmation for the selected generated conformation can often include synthesis when a new molecular entity or new conformation of a molecule.

### EXPERIMENTAL

### Comparative Examples

GraphDG (Simm et al. [2020]) is a CVAE (Sohn et al. [2015]) which models the distribution over distances D given an extended molecular graph G, maximizing the evidence lower bound (ELBO):

L=E_{zq}_{_{Φ}}_{(zD,G)}[log p_{θ}(DZ,G)−D_{KL}[q_{Φ}(zD,G)p_{θ}(zG)]]

Here, p_{θ}(z|G)] is a factorized standard Gaussian distribution. After a pairwise distance matrix is generated, the Euclidean Distance Geometry (EDG) algorithm transforms pairwise distances into conformations.

The CGCF is a conditional graph continuous flow model (Xu [2021]) to learn the factorization of conditional distribution over conformations:

p_{θ}(CG)=∫p(CD,G)p_{θ}(DG)dD

The molecular conformation is obtained by a 3-step process. First, the CGCF model generates a pairwise distance matrix, then the SchNet model refines it, and finally, Euclidean Distance Geometry (EDG) algorithm converts the pairwise distances matrix into a 3D structure. The protocol can approximate the ground truth conformation space p(CIG) with a neural generative model \hat{p}_{θ}(C|G) trained on the set of low-energy molecular conformations (Gᵢ, Cᵢ).

BORSCHT Examples

The BORSCHT protocol evaluates the capacity and quality of the proposed model by performing extensive experiments on the following tasks: (1) Conformation generation—in this task the protocol checks the ability of the model to produce diverse and physically plausible conformations and cover the ground-truth conformations; (2) Conformation modeling with external restrictions—the protocol introduces a novel conformation generation setup for evaluation of the ability of the model to create realistic conformations satisfying given 3D conditions. For all the experiments, the BORSCHT model and optimization hyperparameters are provided herein.

Following the previous works, the protocol performs experiments on GEOM-Drugs and GEOM-QM9 conformation datasets. GEOM dataset (Axelrod et al. [2020]) contains 33 million equilibrium 3D structures computed by xTB+CREST software for 430000 unique molecular graphs. The GEOM-QM9 subset stores reoptimized conformations of small (up to 9 heavy atoms) molecules from QM9 dataset (Ramakrishnan et al. [2014]). The GEOM-Drugs subset contains conformations of medium-sized (up to 91 heavy atoms) drug-like molecules.

In the experiments, the protocol utilized the down-sampled versions of these two subsets. To separate structurally different molecules into train/validation/test subsets, the protocol performs the scaffold split: group conformations by scaffolds, randomly select 10% of scaffolds, divide them into 85%/5%/10% proportions and group conformations by selected scaffolds to obtain corresponding sets. The resulting subsets contain 2608960/178467/317723 conformations for 25252/1656/2910 unique molecular graphs respectively. The proposed model proceed hydrogen-depleted molecular graphs and reduce generating process to producing the Cartesian coordinates only for heavy atoms. The hydrogens Cartesian coordinates of the generated molecules are numerically deduced by running the RDKit software.

The proposed BORSCHT model was analyzed in comparison to three baselines. The experiment trained and evaluated GraphDG and CGCF—two recent state-of-the-art neural generative approaches for molecular conformation modelling. These models generate a matrix of pairwise distances and recover conformations by performing a search of Cartesian coordinates satisfying the given distances. Also, a RDKit conformer generator was adopted, based on MMFF94s algorithm—a popular implementation of rule-based Merck Molecular Force Field.

The goal of this task is to evaluate the ability of the proposed model to generate diverse and physically plausible conformations whose distribution matches the ground truth. For each molecular graph from test sets of GEOM-Drugs and GEOM-QM9 datasets, sample 50 conformations from every model. Let S_{g}(G), Sᵣ(G) further denote the sampled conformation and ground-truth conformations of molecular graph G.

Following the previous works, to measure the dissimilarity between two conformations of a molecular graph G containing n heavy atoms, align conformations and compute the RMSD (Rooted Mean Square Deviation):

$$R M S D ( C , \hat{C} ) = {( \frac{1}{n} {\sum }_{i} {‖ {C}_{i} - {\hat{C}}_{i} ‖}^{2} )}^{1 / 2} \qquad ( 17 )$$

To estimate the diversity of generated conformations compute ICRMSD (Interconformer RMSD) metric—the mean RMSD between all pairs of generated conformation of molecular graph.

$$ICRMSD ( {S}_{ℊ} ( G ) ) = ( \frac{1}{\vert {S}_{ℊ} \cdot ( \vert {S}_{ℊ} \vert - 1 ) \vert} {\sum }_{{C}^{m} ∈ {S}_{ℊ} , {C}^{l} ∈ {S}_{ℊ} , {C}^{m} \neq {equalC}^{l}} R M S D ( {C}^{m} , {C}^{l} ) ) \qquad ( 18 )$$

The protocol assesses the physical plausibility of generated molecules with a computational estimation of potential energy Û(C|G) implemented in MMFF94s algorithm in RDKit software. Accordingly, the RED (Relative Energy Difference) metric—the difference between median potential energies of conformations in generated and ground—is proposed for truth sets, divided by the number n of heavy atoms in molecular graph G.

$$R E D ( {S}_{ℊ} ( G ) , {S}_{r} ( G ) ) = \frac{1}{n} ( {med}_{{C}_{ℊ} ∼ {S}_{ℊ} ( G )} \hat{U} ( {C}_{ℊ} , G ) - {med}_{{C}_{r} ∼ {S}_{r} ( G )} \hat{U} ( {C}_{r} , G ) ) \qquad ( 19 )$$

The COV and MAT metrics are reported, evaluating the similarity between the distribution of generated conformations and the ground-truth distribution. The COV score indicates the percentage of ground truth conformations covered with generated conformation under δ threshold on RMSD. The MAT metrics show how close the ground-truth conformation to the generated conformations in terms of RMSD.

$$COV ( {S}_{ℊ} ( G ) , {S}_{r} ( G ) ) = \frac{1}{\vert {S}_{r} \vert} \vert \{ {C}_{r} ∈ {S}_{r} \vert R M S D ( {C}_{ℊ} , {C}_{r} ) < δ , ∃ {C}_{ℊ} ∈ {S}_{ℊ} \} \vert \qquad ( 20 )$$

$$MAT ( {S}_{ℊ} ( G ) , {S}_{r} ( G ) ) = \frac{1}{\vert {S}_{r} \vert} {\sum }_{{C}_{r} ∈ {S}_{r}} {min}_{{C}_{ℊ} ∈ {S}_{ℊ}} R M S D ( {C}_{ℊ} , {C}_{r} ) \qquad ( 21 )$$

The Table 1 shows that the proposed model is comparable to baselines on COV and MAT metrics on GEOM-QM9 dataset and is better on GEOM-Drugs. Table 1 provides a comparison of the proposed model and baselines on COV and MAT metrics. The values reported on δ=0.5 Å for Geom-QM9 and δ=1.25 Å for Geom-Drugs.

The Table 2 shows that BORSCHT is better than other models on energy-based RED metric and is comparable to RDKit. Table 2 provides a comparison of the proposed model and baselines on ICRMSD and RED metrics.

In this task, the protocol generates a molecular conformations given a values of 3D descriptors. The protocol can modify the proposed model and baselines to take conditions as additional input, proceed with 2-layers MLP and add the transformed condition to the hiddens of all nodes before the first layers of each generative models.

The protocol can evaluate the trained condition model on the test set of Geom-Drugs dataset: for each molecular graph-conformation pair, 3D descriptors of conformation and sample 1 conformation are computed from the model using computed values descriptors and molecular graph as an inputs of models. Next, the protocol evaluates the descriptors of generated molecules and compute for each descriptor the Pearson correlation between condition and resulted values.

In Table 3 the mean and median correlations over 19 descriptors are provided. The proposed BORSCHT model outperforms the baselines with a big gap.

Implementation details for each BORSCHT submodel are provided. All submodels have the same size of hidden states: the node hidden state size is 128, edge hidden state size is 64. Adopt λ_{c}=0.01, λₗ=0.01, λ_{GP}=30 values of coefficients in adversarial optimization problem.

The generator can be implemented to includes a 3-blocks graph embedding part, following by M=4 graph convolution blocks. The model outputs the internal coordinates and distance geometry by applying a 1-blocks graph convolution block and a 2-layer MLP separately. To parametrize distances \hat{l}ₗ, \hat{b}ₗ apply SoftPlus operator to the corresponding outputs of the generator. The sigmoid is applied to the corresponding output of the model and multiply it by π in order to restrict âₗ to be in (0, π).

(h_{emb}ⁿ,h_{emb}^{e})=Emb_{gen}(G)

(hᵢₙₚⁿ,hᵢₙₚ^{e})=(h_{emb}ⁿ+Linear(Z),h_{emb}^{e}+Emb(Eₛ))

(hₒᵤₜⁿ,hₒᵤₜ^{e})=GCB_{gen}^{M}∘ . . . ∘GCB_{gen}¹(hᵢₙₚⁿ,hᵢₙₚ^{e})

\hat{I}=MLP(GCB_{ic}(hₒᵤₜⁿ,hₒᵤₜ^{e}))

\hat{D}=MLP(GCB_{dg}(hₒᵤₜⁿ,hₒᵤₜ^{e}))

The encoder can be implemented to include a 3-blocks graph embedding part, following by P=3 graph convolution blocks and a 2-layer MLP. The protocol applies a dropout layer with 0.1 parameter after each layer of the main body to introduce an additional source of stochasticity. The protocol can apply InstanceNorm on the sampled latent codes to stabilize the training.

(h_{emb}ⁿ,h_{emb}^{e})=Emb_{enc}(G)

(hᵢₙₚⁿ,hᵢₙₚ^{e})=(h_{emb}ⁿ,h_{emb}^{e}+MLP(L)+Emb(Eₛ))

(hₒᵤₜⁿ,*)=GCB_{enc}^{P}∘ . . . ∘GCB_{enc}¹(hᵢₙₚⁿ,hᵢₙₚ^{e})

μ,log σ=MLP(hₒᵤₜⁿ)

Z=μ+e exp(0.5*log σ),e˜N(0,1)

The latent discriminator can be implemented to include a 3-blocks graph embedding part, following by S=3 blocks in the main body and a 2-layer MLP in the end. The protocol can apply a dropout layer with 0.1 parameter after each layer of the main body. The protocol can also set LeakyReLU parameter to 0.2 in the main body and MLP to stabilize the training.

(h_{emb}ⁿ,h_{emb}^{e})=Emb_{latdiscr}(G)

(hᵢₙₚⁿ,hᵢₙₚ^{e})=(h_{emb}ⁿ+Linear(Z),h_{emb}^{e}+Emb(Eₛ))

(hₒᵤₜⁿ,*)=GCB_{latdiscr}^{S}∘ . . . ∘GCB_{latdiscr}¹(hᵢₙₚⁿ,hᵢₙₚ^{e})

aggr=cat(meanpool(hₒᵤₜⁿ),maxpool(hₒᵤₜⁿ))

out=σ(MLP(aggr))

The confirmation discriminator can be implemented to include a 3-layer graph embedding part, I=4 SchNet layers and a 2-layer MLP in the end.

(h_{emb}ⁿ,h_{emb}^{e})=Emb_{confdiscr}(G)

(hₒᵤₜⁿ,*)=SchNetInteractionₗ∘ . . . ∘SchNetInteraction₁(hᵢₙₚ,C)

aggr=cat(meanpool(hₒᵤₜⁿ),maxpool(hₒᵤₜⁿ))

out=MLP(aggr)

The model can be trained as described herein. The training protocol implements the model in PyTorch framework and train on Tesla K80 hardware. Three distinct optimizers can be used to train the model: Adam optimizer with learning rate 0.0003 and betas (0.9, 0.999) for the pair of encoder and generator; Adam optimizer with learning rate 0.0003 and betas (0.5, 0.999) for latent discriminator; Adam optimizer with learning rate 0.001 and betas (0.5, 0.999) for conformation discriminator. The protocol trained the model for 1 epoch on the GEOM-Drugs dataset with batch size 32, and for 40 epochs on GEOM-QM9 dataset with batch size 128. Training procedure takes approximately 4 days for GEOM-Drugs and 2 days for GEOM-QM9.

The protocol adopted 19 descriptors from RDKit software to perform a conditional generation experiment. They contain asphericity, eccentricity, inertial shape factor, two normalized principal moments ratios, three principal moments of inertia, gyration radius, and spherocity index of conformations, computed with and without considering atom masses. The whole list of computed descriptors is given below:

```
from rdkit.Chem import Descriptors3D
def compute_descriptors ( mol ) :
descr_list = [
  Descriptors3D . Asphericity ( mol, 0 , useAtomicMasses=False ) ,
  Descriptors3D . Asphericity ( mol, 0 , useAtomicMasses=True ) ,
  Descriptors3D . Eccentricity ( mol, 0, useAtomicMasses=False ) ,
  Descriptors3D . Eccentricity ( mol, 0, useAtomicMasses=True ) ,
  Descriptors3D . InertialShapeFactor
  ( mol, 0 , useAtomicMasses=False ) ,
  Descriptors3D . InertialShapeFactor
  ( mol, 0 , useAtomicMasses=True ) ,
  Descriptors3D .NPR1( mol , 0 , useAtomicMasses=False ) ,
  Descriptors3D .NPR1( mol ,0 ,useAtomicMasses=True ) ,
  Descriptors3D .NPR2( mol ,0 ,useAtomicMasses=False ) ,
  Descriptors3D .NPR1( mol ,0 ,useAtomicMasses=True ) ,
  Descriptors3D .PMI1( mol ,0 ,useAtomicMasses=False ) ,
  Descriptors3D .PMI1( mol ,0 ,useAtomicMasses=True ) ,
  Descriptors3D .PMI2( mol ,0 ,useAtomicMasses=False ) ,
  Descriptors3D .PMI2( mol ,0 ,useAtomicMasses=True ) ,
  Descriptors3D .PMI3( mol ,0 ,useAtomicMasses=False ) ,
  Descriptors3D .PMI3( mol ,0 ,useAtomicMasses=True ) ,
  Descriptors3D .RadiusOfGyration
  ( mol , 0 , useAtomicMasses=False ) ,
  Descriptors3D . RadiusOfGyration
  ( mol , 0 , useAtomicMasses=True ) ,
  Descriptors3D . SpherocityIndex ( mol , 0)]
return descr_list
```

The protocol can also perform an ablation study and train the only-AAE part and only-GAN variants of the proposed model. In Table 4, COV, MAT, ICRMDS and RED metrics for models were trained on the GEOM-Drugs dataset. The only-AAE model provides diverse conformations, but it is still worse by physical plausibility than the full AAE-GAN model. The only-GAN model is worse than the AAE-GAN model both by diversity and plausibility of generated conformations.

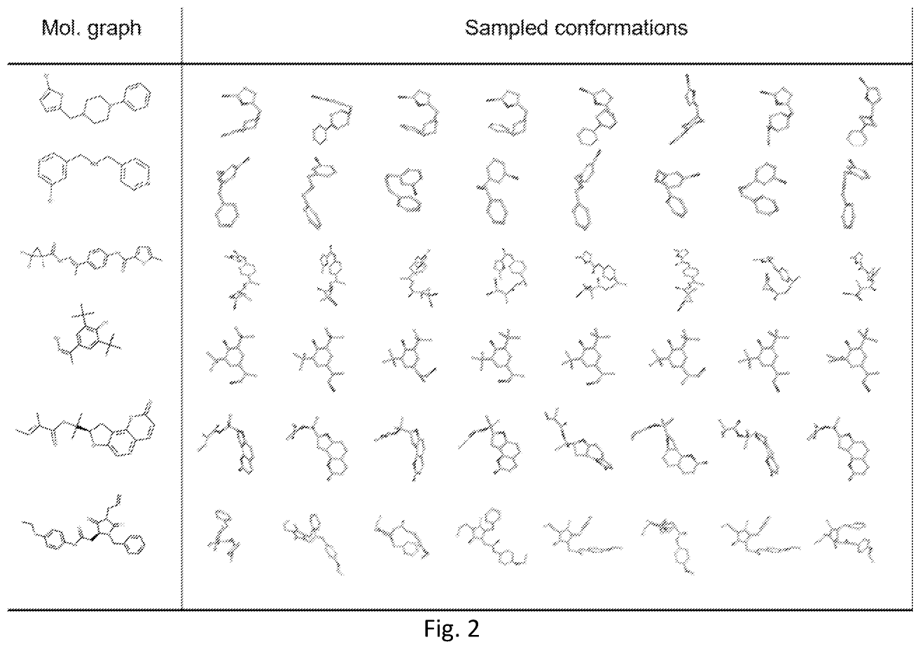

FIG. 2 shows the samples from BORSCHT model trained on the GEOM-Drugs dataset.

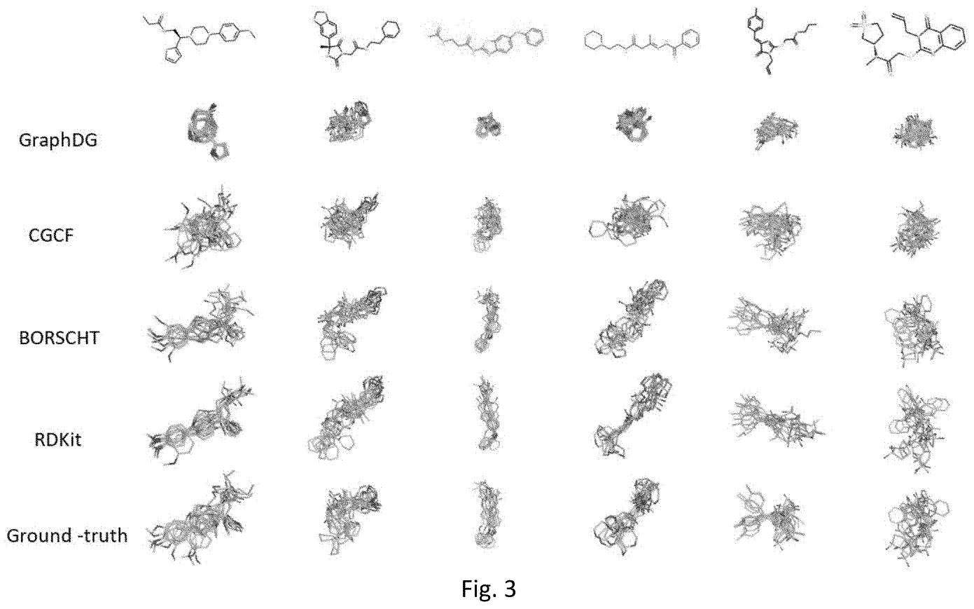

FIG. 3 shows an overlay of 50 conformations from the ground-truth, proposed BORSCHT model and three baseline models for six molecular graphs from the GEOM-Drugs dataset.

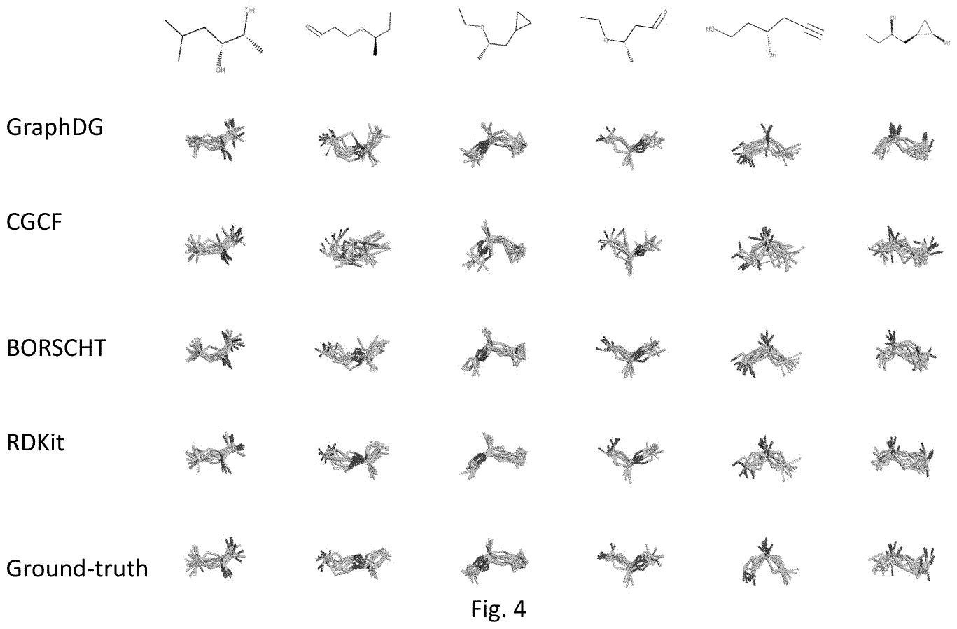

FIG. 4 shows an overlay of 50 conformations from the ground-truth, proposed BORSCHT model and three baseline models for six molecular graphs from GEOM-QM9 dataset.

COSMIC Examples

The capacity and quality of the proposed COSMIC model is evaluated by performing extensive experiments on the following tasks. A conformation generation protocol compares the COSMIC model with state-of-the-art neural approaches and checks the ability of the model to produce diverse and physically plausible conformations and to cover the ground-truth conformations on several conformation datasets. The 3D descriptors-conditioned conformation generation protocol is performed by introducing a novel conditional setup to evaluate the ability of models to create realistic conformations satisfying given 3D descriptors values.

The protocol performs the experiments using GEOM—a well-established conformation dataset, which provides accurate conformations obtained by quantum mechanical methods. GEOM dataset (Axelrod [2020]) and contains two subsets, GEOM-Drugs and GEOM-QM9, with 33 million equilibrium 3D structures computed by xTB+CREST software for 430000 unique molecular graphs. The GEOM-QM9 subset stores reoptimized conformations of small molecules (up to 9 heavy atoms) from the QM9 dataset (Ramakrishnan et al. [2014]). The GEOM-Drugs subset contains conformations of medium-sized drug-like molecules (up to 91 heavy atoms). These two subsets include a good portion of drug-like molecules, which implies generalizability to this crucial class of molecules.

Previous works split these two subsets into train/validation/test sets randomly, which may cause data leaks and compromise test metrics. Unlike predecessors, the protocol introduces a scaffold split to evaluate models' generalizability faithfully. To separate molecules into train/validation/test subsets, perform the following steps: (1) the protocol groups molecules by their Bemis-Murcko scaffolds (Bemis, et al [1996]) of molecular graphs, (2) divide scaffolds into 85%/5%/10% proportions, and (3) collect conformations with selected scaffolds to obtain corresponding train/validation/test sets.

Similar to previous works, the protocol performs a downsampling procedure for both Geom-DRUGS and Geom-QM9 subsets and leave only 10% of scaffolds. Note that since the proposed scaffold data split differs from a random split, the metrics for baselines can differ from the original papers. In the current COSMIC model, the protocol passes hydrogen-depleted molecular graphs and produce the Cartesian coordinates only for heavy atoms. Thus, the protocol numerically deduces the coordinates of the hydrogen atoms by running RDKit.

The protocol evaluates the COSMIC model compared to the recently published neural generative approaches for conformation modeling—GraphDG (Simm et al., [2020]), CGCF (Xu et al. [2021a]), ConfVAE (Xu et al. [2021b]) and GeoMol (Ganea et al. [2021]). Similar to the previous works, the protocol adopts an RDKit conformer generator (Landrum [2006]) in the baselines as an example of traditional non-neural rule-based conformation generators. The protocol obtains the results of all baselines by running their official code.

The validation metrics can be performed. Following the previous works, the protocol computes several metrics to estimate the closeness of the ground-truth [symbol] ^{r} and generated [symbol] ^{g} conformation sets for a molecular graph [symbol] . Given the metrics values for each molecular graph, the protocol then computes the mean and median to obtain final metrics for the whole generated dataset. First, to measure the dissimilarity between two conformations C, Ĉ containing n heavy atoms, the protocol aligns them with roto-translation transformation Φ(C) and compute the RMSD metric (Rooted Mean Square Deviation). The protocol can estimate the spatial diversity of generated conformations by the icRMSD (interconformer RMSD) metric, which computes the mean RMSD between all generated conformations of [symbol] .

$$\begin{matrix}R M S D ( C , \hat{C} ) = {{min}_{Φ} ( \frac{1}{\vert V \vert} {\sum }_{1 \leq i \leq \vert V \vert} {‖ {C}_{i} - Φ ( {\hat{C}}_{i} ) ‖}^{2} )}^{1 / 2} \\ ic R M S D ( {𝕊}^{ℊ} ) = \frac{{\sum }_{{C}^{m} , {C}^{l} ∈ {𝕊}^{ℊ} , {C}^{m} \neq , {C}^{l}} R M S D ( {C}^{m} , {C}^{l} )}{\vert {𝕊}^{ℊ} \vert \cdot ( \vert {𝕊}^{ℊ} \vert - 1 )}\end{matrix} \qquad ( J )$$

The COV and MAT scores are reported to evaluate the quality of coverage of the ground-truth set with generated samples. The COV value indicates the percentage of ground-truth conformations covered with generated conformations under the threshold 8 on RMSD. The MAT metric shows how close the ground-truth conformations are to the generated samples in terms of RMSD.

$$\begin{matrix}COV ( {𝕊}^{ℊ} , {𝕊}^{r} ) = \frac{\vert \{ {C}^{r} ∈ {𝕊}^{r} \vert R M S D ( {C}^{ℊ} , {C}^{r} ) < δ , ∃ {C}^{ℊ} ∈ {𝕊}^{ℊ} \} \vert}{\vert {𝕊}^{r} \vert} \\ MAT ( {𝕊}^{ℊ} , {𝕊}^{r} ) = \frac{1}{\vert {𝕊}^{r} \vert} {\sum }_{{C}^{r} ∈ {𝕊}^{r}} {min}_{{C}^{ℊ} ∈ {𝕊}^{ℊ}} R M S D ( {C}^{ℊ} , {C}^{r} )\end{matrix} \qquad ( K )$$

The inventors propose the RED (Relative Energy Difference) metric—the difference between median potential energies of conformations in generated and ground-truth sets, divided by the number |V| of heavy atoms in the molecular graph [symbol] .

$$R E D ( {𝕊}^{ℊ} , {𝕊}^{r} ) = \frac{1}{\vert V \vert} ( {med}_{{C}^{ℊ} ∼ {𝕊}^{ℊ}} U ( {C}^{ℊ} , 𝒢 ) - {med}_{{C}^{r} ∼ {𝕊}^{r}} U ( {C}^{r} , 𝒢 ) ) \qquad ( L )$$

The protocol can assess the physical plausibility of generated molecules with a computational estimation of potential energy U(C, [symbol] ) implemented in the MMFF94s algorithm in RDKit (Landrum [2006]). One can choose MMFF94s as a good trade-off between computational time and accuracy. However, the invention is not limited the methodology with this choice.

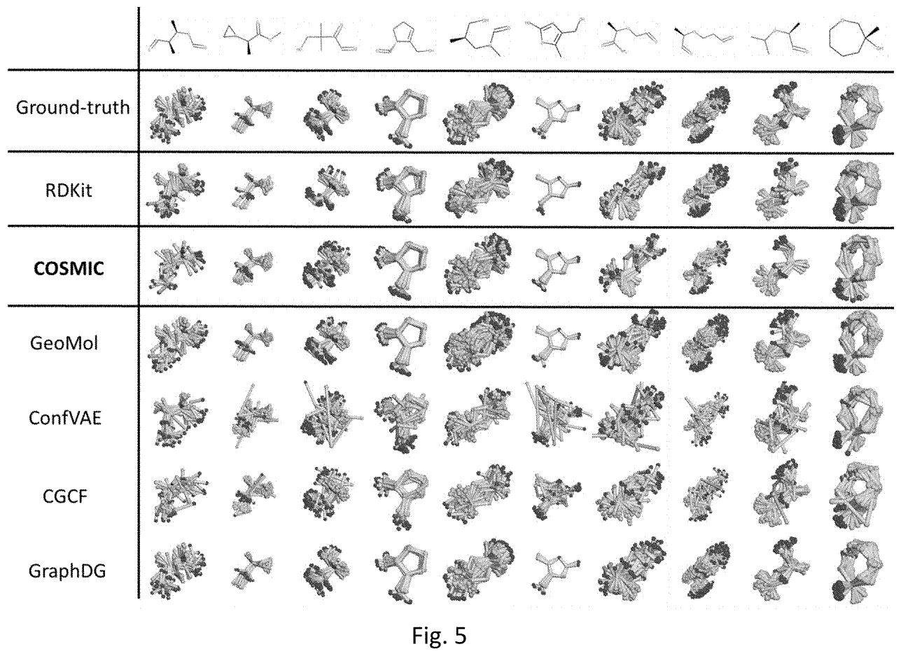

The generations of different conformations of the molecules was performed. This task aims to evaluate the ability of models to generate diverse and physically plausible conformations whose distribution matches the ground truth. The protocol sampled 50 conformations from every model for each molecular graph from the test sets, similar to the previous works. The metrics for the GEOM dataset are in Table. 5 and visualize the generated conformation sets in FIG. 5.

Table 5 provides a comparison of the proposed model and baselines on the GEOM-Drugs and GEOM-QM9 datasets in the unconditional setup. The values of the COV metric are reported for δ=1.25 Å on GEOM-Drugs and δ=0.5 Å on GEOM-QM9.

FIG. 5 shows the visualization of ground-truth and generated conformations by the COSMIC model and baselines. Each part of the visualization contains 10 conformations, where conformations in each column are aligned.

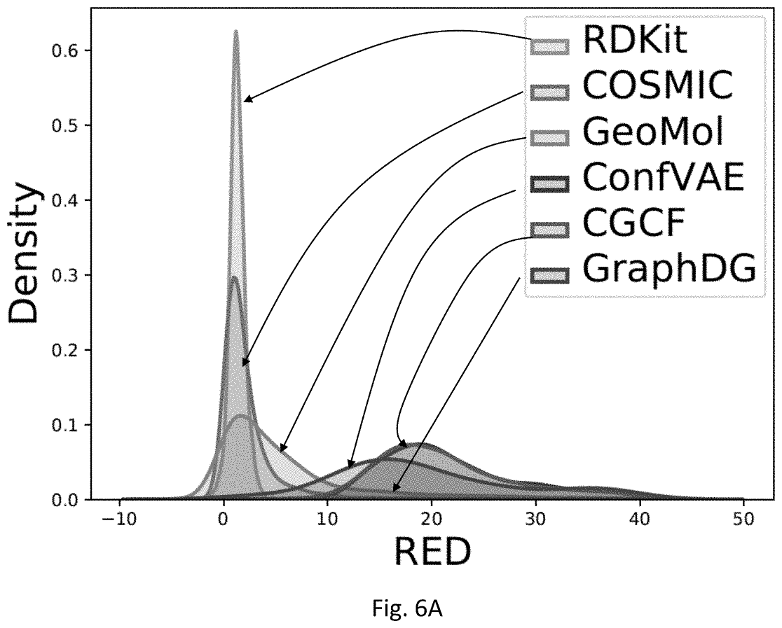

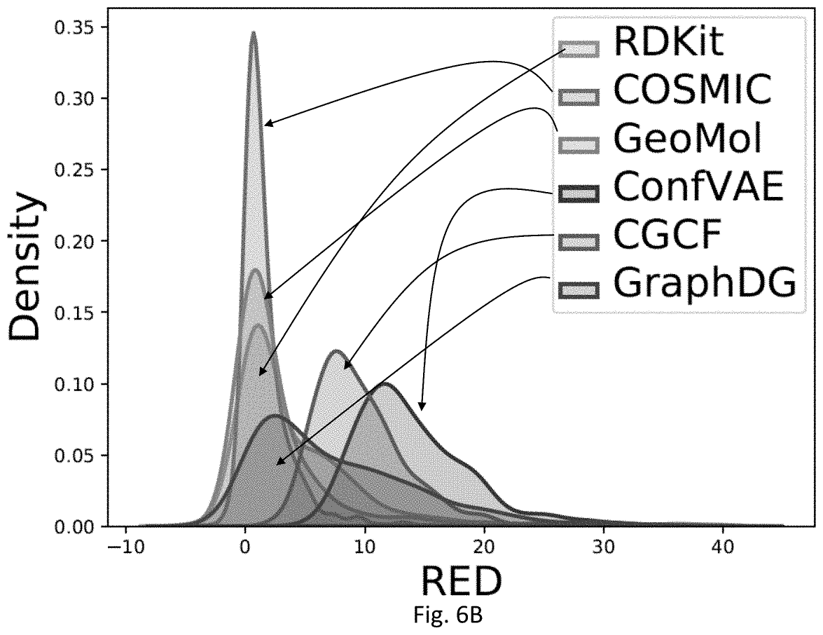

The protocol provides the distribution of RED values in FIGS. 6A-6B for a deeper investigation of conformation plausibility. FIG. 6A shows the RED values distributions on GEOM-DRUGS and FIG. 6B shows the RED values distributions on GEOM-QM9 datasets.

COSMIC outperforms neural-based conformation generators on the GEOM-Drugs dataset on COV, MAT, and with a large margin on the RED metric. Moreover, COSMIC outperforms the RDKit on almost all metrics, losing only on the mean RED metric. Moreover, FIGS. 6A-6B confirm these findings and shows that COSMIC has lighter tails among neural baselines, losing only to rule-based RDKit.

For GEOM-QM9, in Table 5, COSMIC outperforms all models on the median and mean RED values, including non-neural RDKit. Moreover, the distribution of the values in FIGS. 6A-6B are more peaked and have lighter tails of outlier conformations. Among neural-based models for GEOM-QM9, the data shows comparable results on median COV and MAT.

Therefore, the results show that COSMIC outperforms current neural approaches and works on par with RDKit regarding distribution coverage and conformation plausibility. This fact promises that neural-based approaches will replace the traditional rule-based methods for small and medium-size drug-like molecules in the near future.

Conditional conformation modeling was performed. Creating and finding conformations with predefined 3D specifications is one of the essential tasks of the 3D-QSAR (Verma et al. [2010]) approach for drug discovery. The protocol evaluates the ability of models to create realistic conformations satisfying the provided 3D descriptors values.

For example, adopt widely used WHIM (Todeschini et al. [1997]) descriptors as a condition for conformation generation and train models to reconstruct conformations having specific values of these 3D characteristics. WHIM descriptors are roto-translation invariant and contain 114 values describing the 3D properties of the conformation.

The protocol can modify COSMIC and neural-based baselines to take WHIM descriptors as additional input and train models to reconstruct conformations provided their ground-truth descriptor values. Since previous baselines performed only unconditional generation, the protocol modifies all models in the same way to provide a fair comparison. Specifically, the protocol applies a 2-layer MLP to encode WHIM values and add the resulting vector to the node hidden states before the first layers in each part of the generative model. Finally, the protocol omits rule-based baselines such as RDKit because they cannot easily support the conditional generation.

The protocol can evaluate the conditional model on the Geom-Drugs dataset. The protocol can compute WHIM descriptors values for each graph-conformation pair and sample one conformation from the model given these values as the condition. Next, the protocol evaluates the generated conformations' descriptors and compute the descriptor-wise Pearson correlation between WHIM descriptors of the input and generated conformations. Finally, the protocol computes the mean over 114 computed correlations. Also, the protocol can provide the RED metric and RSMD as well. The corresponding metrics values are in Table 6.

In total, the proposed model outperforms all baselines on all metrics with a large margin. This result shows that COSMIC can fully utilize the 3D information to generate desired plausible conformations with specific conditions.

The graph convolution block can be configured as follows. A basic building layer of the COSMIC model is a graph convolutional block (GCB). The protocol can utilize it to update both the representations of nodes hₙ and edges hₑ. The block is based on the Graph Transformer Layer (GTL) of the graph convolution architecture (Shi et al. [2020]) which updates node states hₙ. A multilayer perceptron (MLP) with residual connections is added on top of GTL to additionally update hidden states of edges hₑ. Let h[s] and h[f] denote the operation of taking elements of an array h by the sequence of indices of edge source nodes s and target nodes f respectively, and concat( . . . ) denote matrix concatenation along the feature dimension. The action of the GCB can be summarized in the following way:

hₙ^{t+1}=ReLU(GTL^{l+1}(hₙ^{l},hₑ^{l})) (M)

mₑ^{l}=concat(hₑ^{l},hₙ^{l+1}[s],hₙ^{l+1}[f])

hₑ^{l+1}=ReLU(hₑ^{l}+MLP(mₑ^{l})

The protocol employ this graph convolutional block to construct neural networks of COSMIC modules.

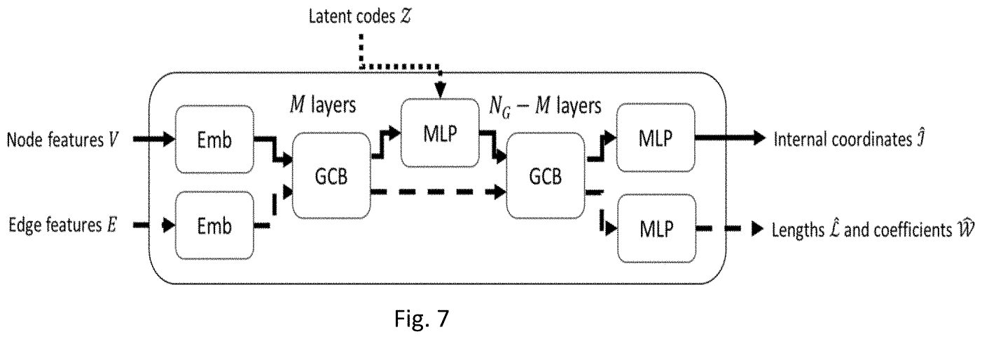

The generator network implementation can be configured as follows. FIG. 7 illustrates a generator network architecture. The generator graph neural network (FIG. 7) is built of several GCBs, introduced in Eq. M, and two MLP heads—one for node-wise internal coordinates [symbol] , and one for bond-wise parameters [symbol] , [symbol] of the EDG problem. The generator consists of N_{G}=10 GCB layers, first M=4 of which do not have access to latent code. The model outputs the internal coordinates and distance geometry by applying a 2-layer MLP separately on embeddings of nodes and edges.

h₀ⁿ=Emb(V); h₀^{e}=Emb(E) (N)

hᵢⁿ,hᵢ^{e}=GCBⁱ(hᵢ₋₁ⁿ,hᵢ₋₁^{e}),1≤i≤M

hᵢⁿ,hᵢ^{e}=GCBⁱ(hᵢ₋₁ⁿ+Lin( [symbol] ,hᵢ₋₁^{e}),M+1≤i≤N_{G}

[symbol]

=MLPⁿ(h_{N}ⁿ)

[symbol]

, [symbol] =MLP^{e}(h_{N}^{e})

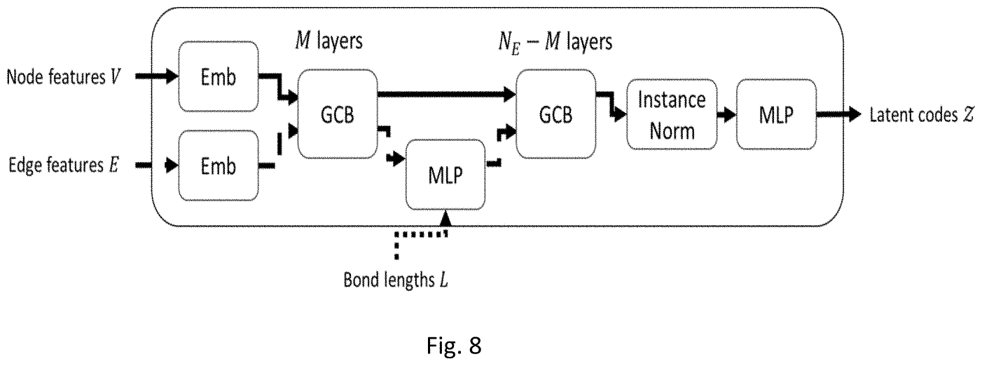

The encoder network implementation can be configured as follows. FIG. 8 illustrates the encoder network architecture. The encoder (FIG. 8) consists of N_{E}=8 blocks totally. Start with M=4 layers, then add information about bond lengths L, and apply 4 GCB layers with a dropout (0.1 ratio) to introduce a source of stochasticity. After, apply InstanceNorm on the node latent codes and finalize with 2-layer MLP.

h₀ⁿ=Emb(V); h₀^{e}=Emb(E) (O)

hᵢⁿ,hᵢ^{e}=GCBⁱ(hᵢ₋₁ⁿ,hᵢ₋₁^{e}),1≤i≤M

h_{M}^{e}=cat(h_{M}^{e},MLP(L))

hᵢⁿ,hᵢ^{e}=GCBⁱ(hᵢ₋₁ⁿ,hᵢ₋₁^{e}),M+1≤i≤N_{E}

[symbol]

=MLP^{enc}(hₚⁿ)

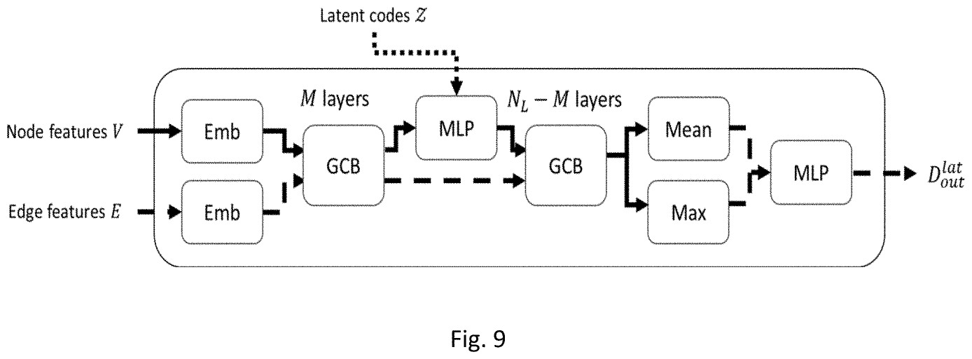

The latent discriminator implementation can be configured as follows. FIG. 9 illustrates the latent discriminator network architecture. Similar to the generator part, the latent discriminator (FIG. 9) consists of N_{L}=8 GCBs. It starts with M=4 GCB layers, then apply 4 GCB layers with a dropout (0.1 ratio) that have access to latent code Z. The network has mean and max-pooling layers applied to node embeddings, followed by 2-layer MLP. Set LeakyReLU parameter to 0.2 in GCB and MLP layers to stabilize the training.

h₀ⁿ=Emb(V); h₀^{e}=Emb(E) (P)

hᵢⁿ,hᵢ^{e}=GCBⁱ(hᵢ₋₁ⁿ,hᵢ₋₁^{e}),1≤i≤M

hᵢⁿ,hᵢ^{e}=GCBⁱ(hᵢ₋₁ⁿ+Lin( [symbol] ),hᵢ₋₁^{e}),M+1≤i≤N_{L}

aggr=cat(meanpool(h_{R}ⁿ),maxpool(h_{R}ⁿ))

Dₒᵤₜ^{lat}=σ(MLP^{lat}(aggr))

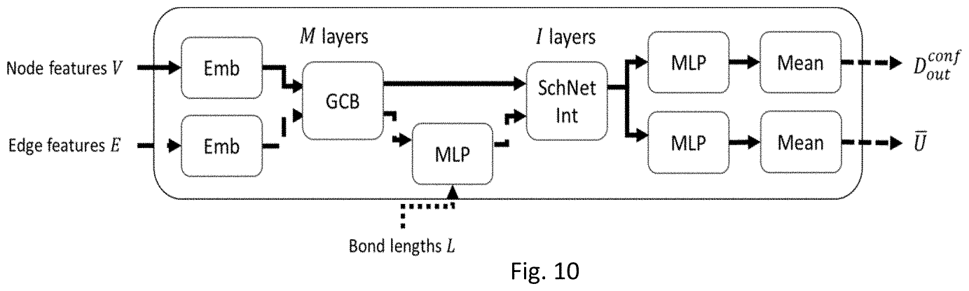

The confirmations discriminator implementation can be configured as follows. FIG. 10 illustrates the confirmations discriminator network architecture. The conformation discriminator (FIG. 10) consists of a M=4 GCB layers, followed by I=4 SchNet interaction layers. The network ends with 2 head, each contains a 2-layer MLP and mean pooling layer in the end.

h₀ⁿ=Emb(V); h₀^{e}=Emb(E) (Q)

hᵢⁿ,hᵢ^{e}=GCBⁱ(hᵢ₋₁ⁿ,hᵢ₋₁^{e}),1≤i≤M

h_{M}^{e}=cat(h_{M}^{e},MLP( [symbol] )

hᵢⁿ,hᵢ^{e}=SchNetIntⁱ(hᵢ₋₁ⁿ,hᵢ₋₁^{e}),M+1≤i≤M+I

Dₒᵤₜ^{conf}=meanpool(MLP^{conf}(h_{M+I}ⁿ))

U̅=meanpool(MLP^{U}(h_{M+I}ⁿ))

Additionally, hyperparameters values and training details are provided. All submodels have the same size of hidden states: the node hidden state size is 128, edge hidden state size is 64. The protocol adopts the following values of coefficients in the training objective: λ_{AAE}=0.01, λ_{WGAN}=0.01, λ_{U}=0.1. Coefficients in reconstruction loss are λ_{D}=100, λ_{I}=50 and gradient penalty coefficient is λ_{GP}=10. The protocol can adopt K=10 iteration of EDG optimization algorithm. Also, a warm-up can be performed: linearly increase coefficients from 0 to stated above values for R=400 steps on GEOM-Drugs and R=100 steps on GEOM-QM9.

The protocol can implement the model in PyTorch framework (Paszke et al. [2019]) and train on Tesla K80 hardware. The protocol uses three distinct optimizers to train the model: Adam optimizer with learning rate 0.0003 and betas (0.9, 0.999) for the pair of encoder and generator; Adam optimizer with learning rate 0.0003 and betas (0.5, 0.999) for latent discriminator; Adam optimizer with learning rate 0.0003 and betas (0.9, 0.999) for conformation discriminator.

The protocol can train the model for 6 epoch on the GEOM-Drugs dataset with batch size 256, and for 60 epochs on GEOM-QM9 dataset with batch size 256. Training procedure takes approximately 3 days for both GEOM-Drugs and GEOM-QM9.

The internal coordinates computations can be performed as described herein. To obtain internal coordinates, one needs to construct a spanning tree of the molecular graph G and assign a graph traversal order starting with a hanging node. Let l, k, j, i be the indices of consecutive nodes in the graph traversal process and C be the Cartesian coordinates of conformation. Then, in terms of unit direction

$$= \frac{{𝒞}_{i} - {𝒞}_{j}}{‖ {𝒞}_{i} - {𝒞}_{j} ‖}$$

and unit normal vectors

$$= \frac{\hat{{u}_{jk}} \times \hat{{u}_{ij}}}{‖ \hat{{u}_{jk}} \times \hat{{u}_{ij}} ‖}$$

internal coordinates are:

bᵢ=∥ [symbol] − [symbol] ∥ (18)

aᵢ=arccos(− [symbol] · [symbol] )

dᵢ=sign( [symbol] · [symbol] )arccos( [symbol] · [symbol] )

The first three nodes in a graph traversal procedure have an insufficient number of predecessors. Therefore, their missing coordinates receive zero values.

The WHIM descriptors can be adopted as described herein. The protocol can adopt 114 WHIM descriptors from RDKit software to perform a conditional generation experiment. The code for computing the whole list of descriptors is given below:

```
import numpy as np
from rdkit import Chem
from rdkit. Chem import rdMolDescriptors, rdMolTransforms
def compute_descriptors(mol):
  mol = Chem.AddHs(mol, addCoords=True)
    rdMolTransforms.CanonicalizeMol(mol, ignoreHs=False)
      return rdMolDescriptors.CalcWHIM(mol, 0)
```

Ablation Study

An ablation study was performed to justify the choice of architecture. In Table 7, provide are COV, MAT, icRMDS and RED metrics on the GEOM-Drugs and GEOMQM9 dataset for proposed model parts with/without WGAN-GP part and with/without AAE part, altered to VAE. Disabling AAE or WGAN-GP parts makes the model worse. The only-AAE model provides diverse conformations but is worse by physical plausibility than the full AAE-GAN model. In contrast, the only-GAN model is worse than the AAE-GAN model both by diversity and plausibility of generated conformations. Changing the AAE part to VAE does not change model performance drastically; both variants have identical metric values.

Table 7 shows the ablation study of the model with disable and altered subparts on the GEOM-Drugs and GEOM-QM9 datasets. The values of the COV metric are reported for δ=1.25 Å on GEOM-Drugs and δ=0.5 Å on GEOM-QM9.

A different number of EDG optimization steps K were also studied, with the results in Table 8. It was found that K=10 is an optimal value based on the tread-off between memory/training time costs and model quality. Table 8 shows the ablation study of the proposed model w.r.t. the number of EDG optimization steps. The values of the COV metric are reported for δ=1.25 Å on GEOM-Drugs and δ=0.5 Å on GEOMQM9.

Finally, in Table 9, investigates how disabling different parts of the training objective changes model performance. Results show that both parts R_{I}, R_{D} of reconstruction loss are essential for model quality. Energy objective does not change distribution coverage metrics but drastically changes the mean RED value—model generates more outliers with high energy without this objective. Table 9 shows the ablation study of the proposed model with disabled parts of the training objective on GEOM-Drugs. The values of the COV metric are reported for δ=1.25 Å.

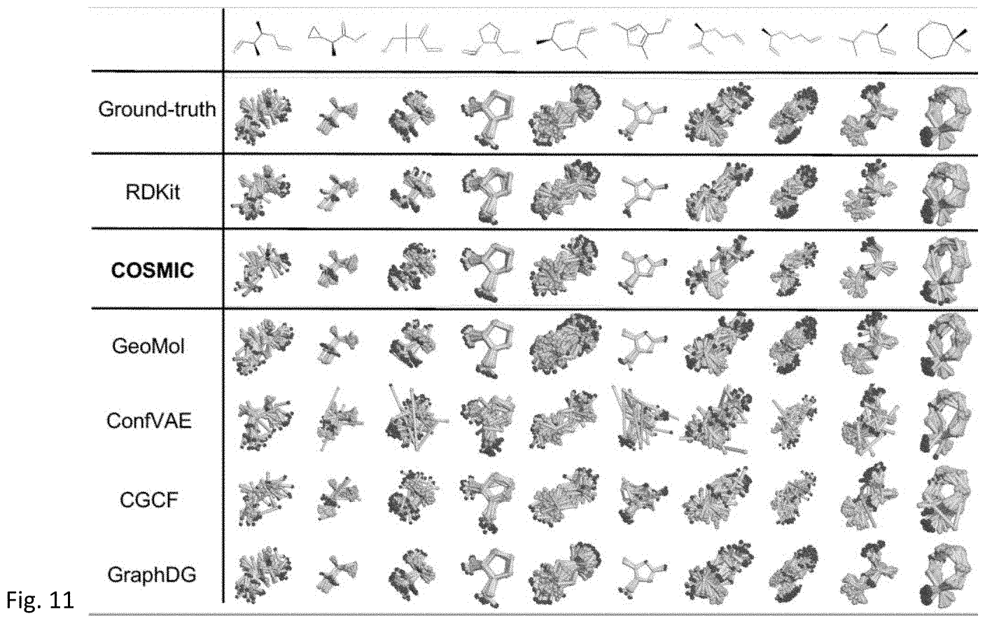

FIG. 11 shows the visualization of ground-truth and generated conformations by COSMIC model and baselines on GEOM-QM9 dataset. Each part of the visualization contains 10 conformations, where conformations in each column are aligned

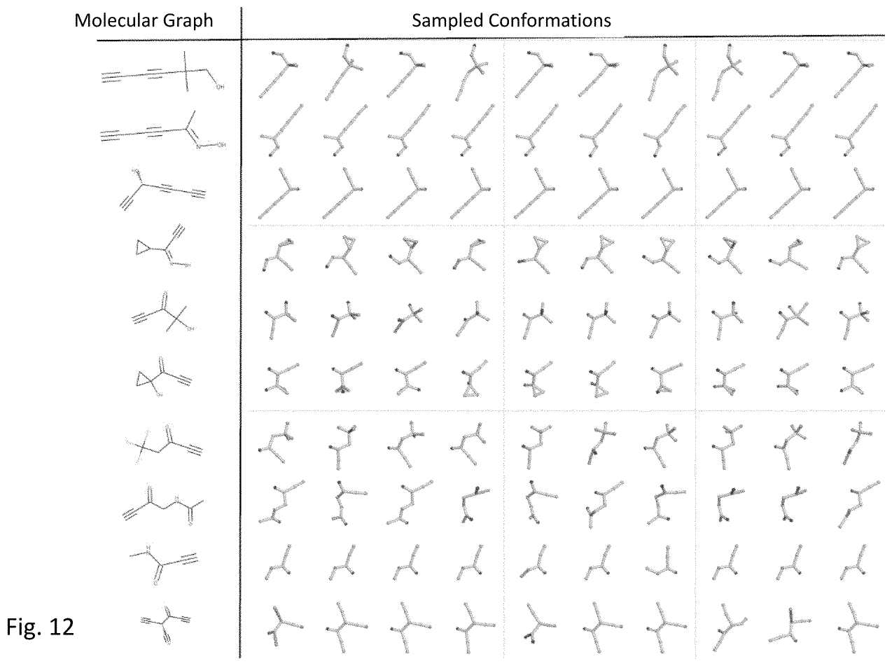

FIG. 12 shows the individual samples from the COSMIC model trained on the GEOM-QM9 dataset.

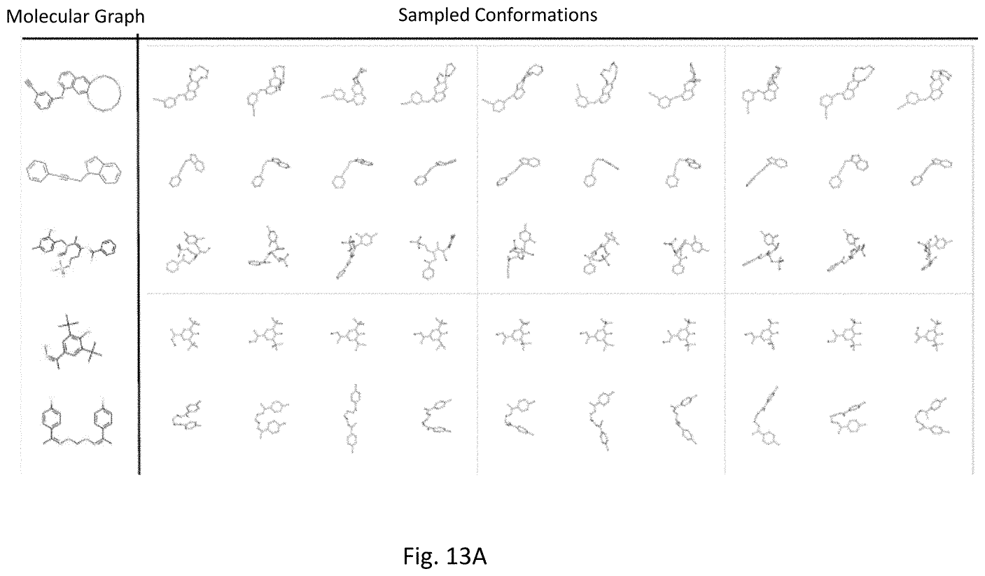

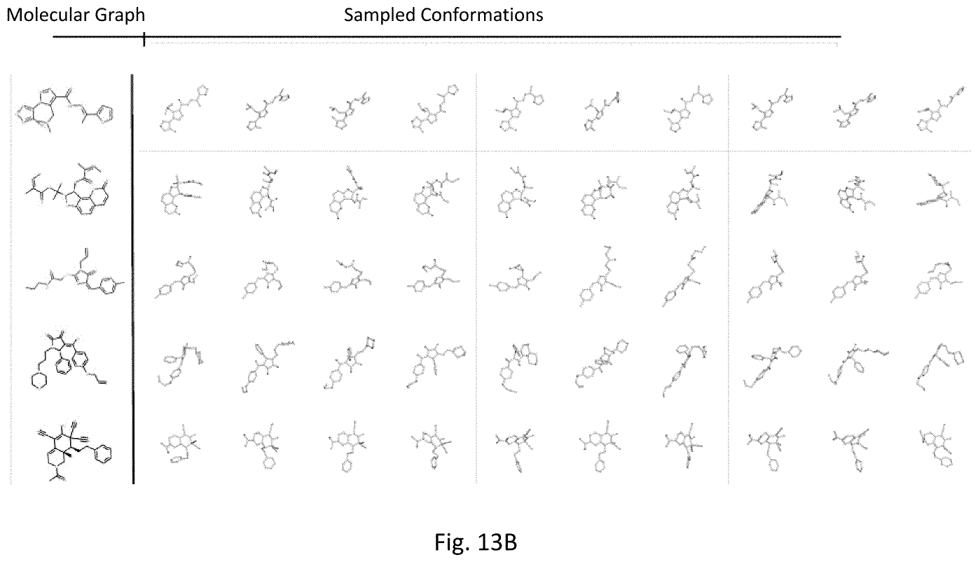

FIGS. 13A-13B show the individual samples from the COSMIC model trained on the GEOM-Drugs dataset.

In traditional computational approaches, the conformation space can be modeled with special software that implements computational methods to approximate the physical forces and quantum effects within the conformation and find an equilibrium state. The methods differ by the level of approximation and computation time—more accurate algorithms require more time to run. The fastest ones iterate over predefined 3D structures of each molecular subfragment and produce combinatorial space of possible conformations (Cole et al. [2018]). The methods based on iterative optimization with rule-based force fields (Halgren [1999]) are more popular in practice since they provide a good trade-off between computational cost and modeling accuracy. Ab initio methods like DFT (Mardirossian et al. [2017]) based on modeling physical and quantum interactions provide the most accurate conformation space estimation while taking a lot of time and resources to run.

The present invention provides a framework for E(3) invariant molecular conformation generation in internal coordinates and introduce a novel metric accounting for conformation energy. Moreover, the invention provides a novel conditional setup to assess the ability of the proposed model and well-established baselines to create conformations satisfying 3D specifications. The experiments show that our approach outperforms the state-of-the-art methods on both unconditional and conditional conformation generation. Future work includes extending the proposed approach to other 3D conditions, such as a protein binding pocket, and increasing the size of the modeled structures.

Deep neural networks (DNNs) are computer system architectures that have recently been created for complex data processing and artificial intelligence (AI). DNNs are machine learning models that employ more than one hidden layer of nonlinear computational units to predict outputs for a set of received inputs. DNNs can be provided in various configurations for various purposes, and continue to be developed to improve performance and predictive ability.

The background architectures can include generative adversarial networks (GANs), which are involved in deep learning to generate novel objects that are indistinguishable from data objects. Conditional GANs or supervised GANs generate objects matching a specific condition.

An autoencoder (AE) is a type of deep neural network (DNN) used in unsupervised learning for efficient information coding. The purpose of an AE is to learn a representation (e.g., encoding) of objects. An AE contains an encoder part, which is a DNN that transforms the input information from the input layer to the latent representation, and includes a decoder part, which decodes an original object with the output layer having the same dimensionality as the input for the encoder. Often, a use of an AE is for learning a representation or encoding for a set of data. An AE learns to compress data from the input layer into a short code, and then uncompress that code into something that closely matches the original data. In one example, the original data may be a molecule that interacts with a target protein, and thereby the AE can design a molecule that is not part of an original set of molecules or select a molecule from the original set of molecules or variation or derivative thereof that interacts (e.g., binds with a binding site) of the target protein.

Generative Adversarial Networks (GANs) are structured probabilistic models that can be used to generate data. GANs can be used to generate data (e.g., a molecule) similar to the dataset (e.g., molecular library) GANs are trained on. A GAN can include two separate modules, which are DNN architectures called: (1) discriminator and (2) generator. The discriminator estimates the probability that a generated product comes from the real dataset, by working to compare a generated product to an original example, and is optimized to distinguish a generated product from the original example. The generator outputs generated products based on the original examples. The generator is trained to generate products that are as real as possible compared to an original example. The generator tries to improve its output in the form of a generated product until the discriminator is unable to distinguish the generated product from the real original example. In one example, an original example can be a molecule of a molecular library of molecules that bind with a protein, and the generated product is a molecule that also can bind with the protein, whether the generated product is a variation of a molecule in the molecular library or a combination of molecules thereof or derivatives thereof.

Adversarial AutoEncoders (AAEs) are probabilistic AEs that use GANs to perform variational inference. AAEs are DNN-based architectures in which latent representations are forced to follow some prior distribution via the discriminator.

Conditional GANs, also referred to as supervised GANS, include specific sets of GAN-based architecture configured for generating objects that match a specific condition. In the usual conditional GAN training process, both the generator and discriminator are conditioned on the same external information (e.g., object and condition pair, such as molecular and target protein pair that bind), which are used during generation of a product.

The model, including the neural networks, can be trained with training datasets in order to be capable of performing the operations described herein. The training procedure includes two steps executed alternately: (1) a generator step; and (2) a discriminator step. A separate objective function is optimized for one optimization step at each update using an optimization method. An Adam optimizer is an example. Training is terminated when the model loss converges or a maximum number of iterations is reached, which can be defined. As such, the iterations can be used to train the neural networks with the training datasets. A result of this training procedure is a generative model, such as the data generator, which is capable of producing new 3D conformations.

The methodologies provided herein can be performed on a computer or in any computing system, such as exemplified in FIG. 6. As such, the computer can include generative adversarial networks that are adapted for conditional generation of molecules (e.g., generated 3D conformations), when a known external variable, such as the condition (e., 3D conformation), influences and improves generation and decoding. The computing system can process the models described herein that are based on the adversarial autoencoder architecture.

One skilled in the art will appreciate that, for the processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In one embodiment, the present methods can include aspects performed on a computing system. As such, the computing system can include a memory device that has the computer-executable instructions for performing the methods. The computer-executable instructions can be part of a computer program product that includes one or more algorithms for performing any of the methods of any of the claims.

In one embodiment, any of the operations, processes, or methods, described herein can be performed or cause to be performed in response to execution of computer-readable instructions stored on a computer-readable medium and executable by one or more processors. The computer-readable instructions can be executed by a processor of a wide range of computing systems from desktop computing systems, portable computing systems, tablet computing systems, hand-held computing systems, as well as network elements, and/or any other computing device. The computer readable medium is not transitory. The computer readable medium is a physical medium having the computer-readable instructions stored therein so as to be physically readable from the physical medium by the computer/processor.

There are various vehicles by which processes and/or systems and/or other technologies described herein can be effected (e.g., hardware, software, and/or firmware), and that the preferred vehicle may vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The various operations described herein can be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware are possible in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a physical signal bearing medium include, but are not limited to, the following: a recordable type medium such as a floppy disk, a hard disk drive (HDD), a compact disc (CD), a digital versatile disc (DVD), a digital tape, a computer memory, or any other physical medium that is not transitory or a transmission. Examples of physical media having computer-readable instructions omit transitory or transmission type media such as a digital and/or an analog communication medium (e.g., a fiber optic cable, a waveguide, a wired communication link, a wireless communication link, etc.).

It is common to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. A typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems, including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. Such depicted architectures are merely exemplary, and that in fact, many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include, but are not limited to: physically mateable and/or physically interacting components and/or wirelessly interactable and/or wirelessly interacting components and/or logically interacting and/or logically interactable components.

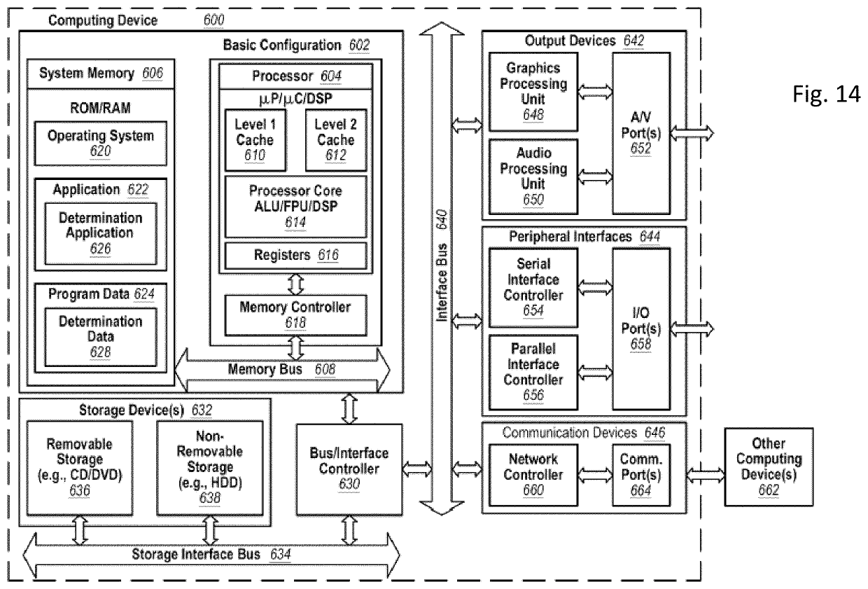

FIG. 14 shows an example computing device 600 (e.g., a computer) that may be arranged in some embodiments to perform the methods (or portions thereof) described herein. In a very basic configuration 602, computing device 600 generally includes one or more processors 604 and a system memory 606. A memory bus 608 may be used for communicating between processor 604 and system memory 606.

Depending on the desired configuration, processor 604 may be of any type including, but not limited to: a microprocessor (p P), a microcontroller (p C), a digital signal processor (DSP), or any combination thereof. Processor 604 may include one or more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations, memory controller 618 may be an internal part of processor 604.

Depending on the desired configuration, system memory 606 may be of any type including, but not limited to: volatile memory (such as RAM), non-volatile memory (such as ROM, flash memory, etc.), or any combination thereof. System memory 606 may include an operating system 620, one or more applications 622, and program data 624. Application 622 may include a determination application 626 that is arranged to perform the operations as described herein, including those described with respect to methods described herein. The determination application 626 can obtain data, such as pressure, flow rate, and/or temperature, and then determine a change to the system to change the pressure, flow rate, and/or temperature.

Computing device 600 may have additional features or functionality, and additional interfaces to facilitate communications between basic configuration 602 and any required devices and interfaces. For example, a bus/interface controller 630 may be used to facilitate communications between basic configuration 602 and one or more data storage devices 632 via a storage interface bus 634. Data storage devices 632 may be removable storage devices 636, non-removable storage devices 638, or a combination thereof. Examples of removable storage and non-removable storage devices include: magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include: volatile and non-volatile, removable and non-removable media implemented in any method or technology for storage of information, such as computer readable instructions, data structures, program modules, or other data.

System memory 606, removable storage devices 636 and non-removable storage devices 638 are examples of computer storage media. Computer storage media includes, but is not limited to: RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 600 may also be implemented as a personal computer including both laptop computer and non-laptop computer configurations. The computing device 600 can also be any type of network computing device. The computing device 600 can also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

In some embodiments, a computer program product can include a non-transient, tangible memory device having computer-executable instructions that when executed by a processor, cause performance of a method that can include: providing a dataset having object data for an object and condition data for a condition; processing the object data of the dataset to obtain latent object data and latent object-condition data with an object encoder; processing the condition data of the dataset to obtain latent condition data and latent condition-object data with a condition encoder; processing the latent object data and the latent object-condition data to obtain generated object data with an object decoder; processing the latent condition data and latent condition-object data to obtain generated condition data with a condition decoder; comparing the latent object-condition data to the latent-condition data to determine a difference; processing the latent object data and latent condition data and one of the latent object-condition data or latent condition-object data with a discriminator to obtain a discriminator value; selecting a selected object from the generated object data based on the generated object data, generated condition data, and the difference between the latent object-condition data and latent condition-object data; and providing the selected object in a report with a recommendation for validation of a physical form of the object. The non-transient, tangible memory device may also have other executable instructions for any of the methods or method steps described herein. Also, the instructions may be instructions to perform a non-computing task, such as synthesis of a molecule and or an experimental protocol for validating the molecule. Other executable instructions may also be provided.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety. References:
Chen, H., et al., The rise of deep learning in drug discovery. Drug Discovery Today, 23(6):1241-1250, 2018a. ISSN 1359-6446. doi: doi.org/10.1016/j.drudis.2018.01.
Vamathevan, J., et al., Applications of machine learning in drug discovery and development. Nat Rev Drug Discov, 18(6):463-477, 06 2019.
Gómez-Bombarelli, R., et al., Automatic Chemical Design Using a Data-Driven Continuous Representation of Molecules. ACS Cent Sci, 4(2):268-276, February 2018.
Zhavoronkov, A. et al., Deep learning enables rapid identification of potent DDR1 kinase inhibitors. Nature biotechnology, pages 1-4, 2019.
Shayakhmetov, R., et al., Molecular generation for desired transcriptome changes with adversarial autoencoders. Frontiers in Pharmacology, 11:269, 2020. ISSN 1663-9812. doi: 10.3389/fphar.2020.00269. (frontiersin.org/article/10.3389/) fphar.2020.00269.
Wu, Z., Ramsundar, B., Feinberg, E., Gomes, J., Geniesse, C., Pappu, A., Leswing, K., and Pande, V. Moleculenet: A benchmark for molecular machine learning. Chemical Science, 9, 03 2017. doi: 10.1039/C7SC02664A.
Gilmer, J. et al., Neural message passing for quantum chemistry, Proceedings of the 34th International Conference on Machine Learning, volume 70 of Proceedings of Machine Learning Research, pages 1263-1272. PMLR, 06-11 Aug. 2017. (proceedings. mlr.press/v70/gilmer17a.html.)
Segler, M. et al., Planning chemical syntheses with deep neural networks and symbolic ai. Nature, 555(7698):604-610, 2018.
Kao, P. et al., Toward drug-target interaction prediction via ensemble modeling and transfer learning. arXiv preprint arXiv:2107.00719, 2021.
Senior, A. et al., Improved protein structure prediction using potentials from deep learning. Nature, pages 1-5, 2020.
Jumper, J., et al. Highly accurate protein structure prediction with alphafold. Nature, 596 (7873):583-589, August 2021. ISSN 1476-4687. doi:10.1038/s41586-021-03819-2. doi.org/10.1038/s41586-021-03819-2.
Ingraham, J., et al., Learning protein structure with a differentiable simulator. In International Conference on Learning Representations, 2019. (openreview.net/forum?id=Byg3y3C9Km).
Van Hilten, N., et al., Virtual compound libraries in computer-assisted drug discovery. Journal of Chemical Information and Modeling, 59(2):644-651, 2019. doi: 10.1021/acs.jcim.8b00737. (doi.org/10.1021/acs.jcim.8b00737).
Blundell, T. L., et al., High-throughput crystallography for lead discovery in drug design. Nature Reviews Drug Discovery, 1(1):45-54, 2002.
Pellecchia, M., et al., Perspectives on NMR in drug discovery: a technique comes of age. Nature reviews Drug discovery, 7(9):738-745, 2008.
De Vivo, M., et al., Role of molecular dynamics and related methods in drug discovery. Journal of Medicinal Chemistry, 59(9):4035-4061, 2016. doi: 10.1021/acs.jmedchem.5b01684. (doi.org/10.1021/acs. jmedchem.5b01684). PMID: 26807648.
Mardirossian, N., et al., Thirty years of density functional theory in computational chemistry: an overview and extensive assessment of 200 density functionals. Molecular Physics, 115(19):2315-2372, 2017. doi: 10.1080/00268976.2017.1333644. (doi.org/10.1080/00268976.2017.1333644).
Halgren, T., Mmff vi. mmff94s option for energy minimization studies. Journal of Computational Chemistry, 20(7):720-729, 1999. doi.org/10.1002/(SICI)1096-987X(199905)20: 7<720::AID-JCC7>3.0.CO;2-X. onlinelibrary.wiley.com/doi/abs/10. 1002/%28SICI%291096-987X%28199905%2920%3A7%3C720%3A%3AAID-JCC7%3E3.0.CO%3B2-X.
Dral, P., Quantum chemistry in the age of machine learning. The journal of physical chemistry letters, 11(6): 2336-2347, 2020.
Mansimov, E., et al., Molecular geometry prediction using a deep generative graph neural network. arXiv preprint arXiv:1904.00314, 2019.
Xu, M., et al., Learning neural generative dynamics for molecular conformation generation. In International Conference on Learning Representations, 2021a. openreview.net/forum?id=pAbmlqfheGk.
Xu, M., et al., An end-to-end framework for molecular conformation generation via bilevel programming. Proceedings of the 38th International Conference on Machine Learning, volume 139 of Proceedings of Machine Learning Research, pp. 11537-11547. PMLR, 18-24 Jul. 2021b. proceedings.mlr. press/v139/xu21f.html.
Simm, G., et al., Reinforcement learning for molecular design guided by quantum mechanics. In III, H. D. and Singh, A. (eds.), Proceedings of the 37th International Conference on Machine Learning, volume 119 of Proceedings of Machine Learning Research, pp. 8959-8969. PMLR, 13-18 Jul. 2020. proceedings.mlr.press/v119/simm20b.html.
Schütt, K., et al., Schnet—a deep learning architecture for molecules and materials. The Journal of Chemical Physics, 148(24): 241722, 2018. doi: 10.1063/1.5019779. doi.org/10.1063/1.5019779.
Smith, J. S., et al., Ani-1: an extensible neural network potential with dft accuracy at force field computational cost. Chemical science, 8(4): 3192-3203, 2017.
Kombo, D. C., et al., 3D molecular descriptors important for clinical success. J Chem Inf Model, 53(2):327-342, February 2013.
Mason, J. S., et al, 3-D pharmacophores in drug discovery. Curr Pharm Des, 7(7): 567-597, May 2001.
Cole, J. C., et al., Knowledge-based conformer generation using the cambridge structural database. Journal of Chemical Information and Modeling, 58(3):615-629, 2018. doi: 10.1021/acs.jcim.7b00697. doi.org/10.1021/acs.jcim.7b00697. PMID: 29425456.
Westermayr, J., et al., Combining schnet and sharc: The schnarc machine learning approach for excited-state dynamics. The Journal of Physical Chemistry Letters, 11(10):3828-3834, April 2020. ISSN 1948-7185. doi: 10.1021/acs.jpclett. 0c00527. dx.doi.org/10.1021/acs.jpclett.0c00527.
Shi, C., et al., Learning gradient fields for molecular conformation generation. Proceedings of the 38th International Conference on Machine Learning, volume 139 of Proceedings of Machine Learning Research, pp. 9558-9568. PMLR, 18-24 Jul. 2021. proceedings.mlr.press/v139/shi21b.html.
Liberti, L., et al., Euclidean distance geometry and applications. SIAM Review, 56, 05 2012. doi: 10.1137/120875909.
Simm, G. N. C., et al., A generative model for molecular distance geometry. III, H. D. and Singh, A. (eds.), Proceedings of the 37th International Conference on Machine Learning, volume 119 of Proceedings of Machine Learning Research, pp. 8949-8958. PMLR, 13-18 Jul. 2020. proceedings.mlr.press/v119/simm20a.html.
Greg Landrum. Rdkit: Open-source cheminformatics software. 2016. github.com/rdkit/rdkit/releases/tag/Release_2016_09_4.
Arjovsky, M., et al, Wasserstein GAN, 2017.
Gulrajani, I., et al., Improved training of wasserstein GANs, 2017.
Makhzani, A., et al., Adversarial autoencoders. CoRR, abs/1511.05644, 2015. arxiv.org/abs/1511.05644.
Shi, Y., et al., Masked label prediction: Unified massage passing model for semi-supervised classification. CoRR, abs/2009.03509, 2020. arxiv.org/abs/2009.03509.
Goodfellow, I., et al., Generative Adversarial Nets. Advances in neural information processing systems, pages 2672-2680, 2014.
Zhou, Z., et al., Lipschitz generative adversarial nets. In International Conference on Machine Learning, pages 7584-7593. PMLR, 2019.
Sohn, K., et al., Learning structured output representation using deep conditional generative models. In C. Cortes, N. Lawrence, D. Lee, M. Sugiyama, and R. Garnett, editors, Advances in Neural Information Processing Systems, volume 28. Curran Associates, Inc., 2015. proceedings.neurips.cc/paper/2015/file/8d55a249e6baa5c06772297520da2051-Paper.pdf.
Axelrod, S., et al., Geom: Energy-annotated molecular conformations for property prediction and molecular generation. arXiv preprint arXiv:2006.05531, 2020.
Ramakrishnan, R., et al., Quantum chemistry structures and properties of 134 kilo molecules. Scientific data, 1:140022, 2014.
Bemis, G., et al., The properties of known drugs. 1. molecular frameworks. J. Med. Chem., 39(15):2887-2893, July 1996.
Ganea, O.-E., et al., Torsional geometric generation of molecular 3d conformer ensembles, 2021.
Verma, J., et al., 3d-qsar in drug design-a review. Current topics in medicinal chemistry, 10(1):95-115, 2010.
Todeschini, R., et al., The whim theory: New 3d-molecular descriptors for qsar in environmental modelling. Sar and Qsar in Environmental Research, 7:89-115, 1997.
Paszke, A., et al., Pytorch: An imperative style, high-performance deep learning library. Advances in Neural Information Processing Systems 32, pages 8024-8035. Curran Associates, Inc., 2019. papers.neurips.cc/paper/9015-pytorch-an-imperative-style-high-performance-deep-learning-library. pdf.
Cole, J., et al., Knowledge-based conformer generation using the cambridge structural database. Journal of Chemical Information and Modeling, 58(3):615-629, 2018. doi: 10.1021/acs.jcim.7b00697. doi.org/10.1021/acs.jcim.7b00697. PMID: 29425456.
Simm, G., et al., A generative model for molecular distance geometry. Proceedings of the 37th International Conference on Machine Learning, volume 119 of Proceedings of Machine Learning Research, pages 8949-8958. PMLR, 13-18 Jul. 2020. proceedings.mlr.press/v119/simm20a.html.
Axelrod, S., et al., Geom: Energy-annotated molecular conformations for property prediction and molecular generation. arXiv preprint arXiv:2006.05531, 2020.

TABLE 1
Shows that the proposed model is comparable to baselines on COV and MAT
metrics on GEOM-QM9 dataset and is better on GEOM-Drugs.

| | QM9 | | | | Drugs | | | |
|---|---|---|---|---|---|---|---|---|
| | COV | | MAT | | COV | | MAT | |
| | Mean | Median | Mean | Median | Mean | Median | Mean | Median |
| GraphDG | 0.0086 | 0.0000 | 0.9809 | 0.9863 | 0.4363 | 0.3500 | 1.433 | 1.3469 |
| CGCF | 0.8684 | 1.0000 | 0.3027 | 0.2840 | 0.5397 | 0.5670 | 1.2632 | 1.2262 |
| BORSCHT | 0.8026 | 1.0000 | 0.2988 | 0.2571 | 0.7686 | 0.9286 | 0.9266 | 0.9131 |
| Rd kit | 0.8892 | 1.0000 | 0.2032 | 0.1361 | 0.6129 | 0.7021 | 1.2200 | 1.061 |

TABLE 2
Shows that BORSCHT is better than other models on energy-based RED metric and is
comparable to RD Kit.

| | QM9 | | | Drugs | | |
|---|---|---|---|---|---|---|
| | ICRMSD | RED | | ICRMSD | RED | |
| | Mean | Mean | Median | Mean | Mean | Median |
| GraphDG | 1.313 | 11486.632 | 9623.760 | 1.231 | 15910.208 | 11079.496 |
| CGCF | 0.996 | 14.719 | 14.444 | 2.336 | 452.322 | 31.555 |
| BORSCHT | 0.752 | 5.270 | 3.714 | 1.966 | 16.556 | 3.339 |
| Rd kit | 0.537 | 6.368 | 1.597 | 2.488 | 26.878 | 1.549 |

TABLE 3
Shows the mean and median of correlations between
conditions and 3D descriptors of generated molecules

| | Mean | Median |
|---|---|---|
| GraphDG | 0.311 | 0.172 |
| CGCF | 0.359 | 0.259 |
| BORSCHT | 0.772 | 0.824 |

TABLE 4
Shows a comparison of the full AAE-GAN proposed model and
the only-GAN, only-AAE variants by COV and MAT metrics.
The values reported on δ = 1.25 Å for Geom-Drugs.

| | COV | | MAT | | ICRMSD | RED | |
|---|---|---|---|---|---|---|---|
| | Mean | Median | Mean | Median | Mean | Mean | Median |
| BORSCHT(AAE) | 0.584 | 0.684 | 1.158 | 1.130 | 1.950 | 14.997 | 5.920 |
| BORSCHT(GAN) | 0.170 | 0.063 | 1.911 | 1.900 | 0.169 | 138.42 | 8.822 |
| BORSCHT(AAE-GAN) | 0.769 | 0.929 | 0.927 | 0.913 | 1.966 | 16.556 | 3.339 |

TABLE 5
Comparison of proposed models.

| COV | | MAT | | icRMSD | | RED | |
|---|---|---|---|---|---|---|---|
| Mean | Median | Mean | Median | Mean | Median | Mean | Median |

GEOM-Drugs

| | | | | | | | | |
|---|---|---|---|---|---|---|---|---|
| GraphDG | 0.4363 | 0.3500 | 1.433 | 1.3469 | 1.198 | 1.035 | 15910.208 | 11079.496 |
| CGCF | 0.5397 | 0.5670 | 1.2632 | 1.2262 | 2.326 | 2.339 | 452.322 | 31.555 |
| ConfVAE | 0.4303 | 0.3125 | 1.3675 | 1.3400 | 2.487 | 2.514 | 639.831 | 62.885 |
| GeoMol | 0.0967 | 0.0000 | 2.5341 | 2.5691 | 2.009 | 1.966 | 67.871 | 4.224 |
| COSMIC | 0.7184 | 0.8571 | 0.9798 | 0.9595 | 1.906 | 1.901 | 7.827 | 1.396 |
| RDKit | 0.5650 | 0.6000 | 1.2842 | 1.1834 | 1.795 | 1.773 | 1.337 | 1.549 |

GEOM-QM9

| | | | | | | | | |
|---|---|---|---|---|---|---|---|---|
| GraphDG | 0.0086 | 0.0000 | 0.9809 | 0.9863 | 0.987 | 0.952 | 11486.632 | 9623.760 |
| CGCF | 0.8684 | 1.0000 | 0.3027 | 0.2840 | 0.996 | 0.952 | 14.719 | 14.444 |
| ConfVAE | 0.7721 | 1.0000 | 0.3741 | 0.3629 | 1.025 | 0.990 | 14.282 | 12.976 |
| GeoMol | 0.2729 | 0.0000 | 0.9729 | 1.0414 | 0.973 | 0.986 | 86.316 | 1.288 |
| COSMIC | 0.7218 | 1.0000 | 0.3313 | 0.2572 | 0.6739 | 0.6470 | 2.123 | 1.004 |
| RDKit | 0.8892 | 1.0000 | 0.2032 | 0.1361 | 0.537 | 0.521 | 6.368 | 1.597 |

TABLE 6
The correlations, RED, and pairwise RMSD
metrics in the conditional setup.

| | | RED ↓ | | RMSD ↓ | |
|---|---|---|---|---|---|
| | Corr ↑ | Mean | Median | Mean | Median |
| GraphDG | 0.285 | 8250 | 5524 | 3.377 | 3.340 |
| CGCF | 0.360 | 1969 | 568 | 2.870 | 2.861 |
| ConfVAE | 0.305 | 2212 | 1203 | 3.045 | 3.046 |
| GeoMol | 0.480 | 934 | 33.51 | 3.340 | 3.282 |
| COSMIC | 0.677 | 4.696 | 1.323 | 2.189 | 2.114 |

TABLE 7
Ablation study of the model with disable and altered subparts
on the GEOM-Drugs and GEOM-QM9 datasets.

| COV | | MAT | | icRMSD | | RED | |
|---|---|---|---|---|---|---|---|
| Mean | Median | Mean | Median | Mean | Median | Mean | Median |

GEOM-Drugs

| | | | | | | | | |
|---|---|---|---|---|---|---|---|---|
| Only AAE | 0.692 | 0.840 | 1.005 | 0.991 | 1.966 | 1.962 | 13.255 | 1.687 |
| Only VAE | 0.670 | 0.800 | 1.035 | 1.037 | 2.000 | 1.982 | 9.402 | 2.025 |
| Only WGAN-GP | 0.449 | 0.407 | 1.346 | 1.324 | 1.312 | 1.210 | 35.925 | 3.647 |
| VAE + WGAN-GP | 0.720 | 0.849 | 0.978 | 0.966 | 1.868 | 1.853 | 8.390 | 1.232 |
| AAE + WGAN-GP | 0.718 | 0.857 | 0.980 | 0.960 | 1.906 | 1.901 | 7.828 | 1.397 |

GEOM-QM9

| | | | | | | | | |
|---|---|---|---|---|---|---|---|---|
| Only AAE | 0.717 | 1.000 | 1.083 | 0.271 | 0.661 | 0.636 | 2.012 | 1.596 |
| Only VAE | 0.762 | 1.000 | 0.306 | 0.259 | 0.755 | 0.740 | 2.841 | 2.055 |
| Only WGAN-GP | 0.500 | 0.500 | 0.515 | 0.502 | 0.415 | 0.205 | 4.922 | 2.300 |
| VAE + WGAN-GP | 0.727 | 1.000 | 0.326 | 0.264 | 0.689 | 0.665 | 2.009 | 1.117 |
| AAE + WGAN-GP | 0.723 | 1.000 | 0.326 | 0.264 | 0.668 | 0.644 | 2.123 | 1.004 |

TABLE 8
Ablation study of the proposed model w.r.t. the number of EDG optimization steps.

| COV | | MAT | | icRMSD | | RED | |
|---|---|---|---|---|---|---|---|
| Mean | Median | Mean | Median | Mean | Median | Mean | Median |

GEOM-Drugs

| | | | | | | | | |
|---|---|---|---|---|---|---|---|---|
| 0 steps | 0.699 | 0.829 | 1.001 | 0.984 | 1.908 | 1.888 | 165.519 | 1.795 |
| 5 steps | 0.709 | 0.846 | 0.984 | 0.969 | 1.904 | 1.891 | 23.714 | 1.165 |
| 10 steps | 0.718 | 0.857 | 0.980 | 0.960 | 1.906 | 1.901 | 7.828 | 1.397 |
| 20 steps | 0.709 | 0.840 | 0.987 | 0.973 | 1.912 | 1.906 | 3.365 | 0.988 |

GEOM-QM9

| | | | | | | | | |
|---|---|---|---|---|---|---|---|---|
| 0 steps | 0.664 | 0.889 | 0.379 | 0.319 | 0.622 | 0.579 | 252.872 | 1.517 |
| 5 steps | 0.714 | 1.000 | 0.333 | 0.264 | 0.653 | 0.626 | 2.067 | 1.012 |
| 10 steps | 0.723 | 1.000 | 0.326 | 0.265 | 0.668 | 0.644 | 2.123 | 1.004 |
| 20 steps | 0.702 | 1.000 | 0.335 | 0.250 | 0.633 | 0.606 | 2.568 | 0.744 |

TABLE 9
Ablation study of the proposed model with disabled parts of the training
objective on GEOM-Drugs.

| | COV | | MAT | | icRMSD | | RED | |
|---|---|---|---|---|---|---|---|---|
| | Mean | Median | Mean | Median | Mean | Median | Mean | Median |
| No L_{U} objective | 0.718 | 0.857 | 0.980 | 0.968 | 1.916 | 1.906 | 18.609 | 1.442 |
| No R_{D} loss | 0.670 | 0.789 | 1.042 | 1.023 | 1.941 | 1.931 | 36.785 | 3.527 |
| NoR_{I} loss | 0.220 | 0.109 | 1.750 | 1.752 | 0.347 | 0.252 | 32.332 | 7.048 |
| Full loss | 0.718 | 0.857 | 0.980 | 0.960 | 1.906 | 1.901 | 7.828 | 1.397 |

1. A computer-implemented method, comprising:
obtaining molecule graph data for a molecule;
inputting the molecule graph data into a machine learning platform;
generating a plurality of conformations for the molecule with the machine learning platform, wherein the plurality conformations are specific to the molecule, each conformation having internal coordinates defining positions of atoms of the molecule;
selecting at least one conformation for the molecule based on at least one parameter related to molecular conformations; and
preparing a report that includes the selected at least one conformation for the molecule.

2. The method of claim 1, further comprising the machine learning platform predicting lengths for each molecular graph bond of the molecule for each conformation.

3. The method of claim 1, wherein the at least one parameter related to molecular conformations includes an energy of each conformation, the method comprising providing the at least one selected conformation of the molecule that has a lower energy compared to other generated conformations of the molecule.

4. The method of claim 1, further comprising the report including a conformation space that is comprised of a plurality of overlaid selected conformations for the molecule.

5. The method of claim 1, further comprising:
inputting molecule graph data of the molecule and a set of latent vectors into a generator;
outputting a conformation of the molecule as a sequence of internal coordinates;
distinguishing real conformations from generated conformations with predicted energy differences;
mapping conformations into latent space; and
conforming the latent space to be similar to a prior distribution.

6. The method of claim 1, further comprising a conformation generation:
generating internal coordinates of a first conformation from the molecule graph data and noise;
predicting bond lengths and a bond-wise loss function weight of the first conformation;
converting the internal coordinates to Cartesian coordinates for the first conformation;
computing the Cartesian coordinates for unit direction and unit normal vectors for the conformation; and
modulating bond length of the conformation to the predicted bond lengths.

7. The method of claim 1, further comprising:
representing the molecular graph by nodes and edge feature sets;
extending the molecular graph with auxiliary nodes and edges to make a proposed generative model;
introducing virtual edges between second, third, and/or fourth neighboring nodes;
setting each node to include a description of: atom type, charge, and chiral tag;
setting each edge feature to include a first graph subset that has chemical bond type and bond stereochemistry; and
setting each edge feature to include a second graph subset that has a spanning tree traversal process and having defining edge features to be in the spanning tree and information regarding whether a source node appears earlier in the spanning tree traversal process than a destination node.

8. The method of claim 1, further comprising estimating one or more of the following conformation properties for each generated molecule: asphericity, eccentricity, inertial shape factor, two normalized principal moments ratios, three principal moments of inertia, gyration radius or spherocity index.

9. The method of claim 1, further comprising:
operating a molecular graph generator to obtain molecular graph data and latent code data to construct a conformation of a molecule with a set of internal coordinates, to convert the internal coordinates into Cartesian coordinates, and perform at least one optimization to correct local distance geometry of at least one molecular substructure;
operating a conformation discriminator to distinguish between real conformations of a molecule from synthetic conformations of the molecule;
operating a stochastic encoder to construct an irredundant latent space of latent data of input molecules and prevent mode collapse; and
operating a latent variables discriminator to map conformations into the latent space and to make the latent space similar to a normal prior distribution.

10. The method of claim 1, further comprising determining a reconstruction loss between an original conformation of a molecule compared to a reconstructed conformation of the molecule by adversarial analysis between the molecular graph generator against the confirmation discriminator and latent variables discriminator.

11. The method of claim 1, further comprising:
constructing a first conformation having a rotation and translation invariant representation; and
predicting distances between neighboring atoms of the first confirmation.

12. The method of claim 1, further comprising:
considering a potential energy of a plurality of conformations; and
selecting physically plausible conformations based on the potential energy of each selected conformation.

13. The method of claim 1, further comprising:
modeling at least one provided conformation of the molecule with a biological target; and
determine whether or not the at least one provided conformation modulates the biological target.

14. The method of claim 1, further comprising:
operating a graph convolution block to:
update representations of nodes and edges of a molecule graph data;
update node states; and/or
update hidden states of edges.

15. The method of claim 1, further comprising inputting condition data into the machine learning platform, wherein the condition data is at least one conformation of the molecule.

16. The method of claim 1, further comprising:
encoding discrete features of nodes and edge features with embedding layers, each edge feature including a first graph subset that has a chemical bond type and bond stereochemistry; and
applying a sequence of graph convolution blocks to the discrete features to obtain an embedding of the molecular graph of the molecule.

17. The method of claim 1, further comprising an encoder:
obtaining a description of a conformation from molecular graph data of a molecule; and
transforming the conformation with a sequence of graph convolution blocks to obtain node-wise latent codes,
wherein the latent codes are stochastic and sampled with reparameterization from a normal distribution parameterized by outputs of the encoder.

18. The method of claim 1, further comprising a latent variables discriminator:
distinguishing generated latent codes of real conformations from noise; and
determining:
node-wise latent codes being independent of each other; and
node-wise latent codes following the normal distribution.

19. The method of claim 1, further comprising a conformation discriminator:
controlling quality of generated objects by:
assessing a likelihood of one or more conformations;
determining a quality of the one or more conformations based on potential energy estimations.

20. The method of claim 1, further comprising a conformation discriminator:
passing molecular graph embeddings through a plurality of SchNet layers to obtain node representations; and
obtaining one aggregated value for the whole molecular conformation.

21. The method of claim 1, further comprising determining an ability to synthesize generated molecular conformation, wherein the generated molecular conformation has at least one three dimensional restriction.

22. One or more non-transitory computer readable media storing instructions that in response to being executed by one or more processors, cause a computer system to perform operations, the operations comprising:
obtaining molecule graph data for a molecule;
inputting the molecule graph data into a machine learning platform;
generating a plurality of conformations for the molecule with the machine learning platform, wherein the plurality conformations are specific to molecule, each conformation having internal coordinates defining positions of atoms of the molecule;
selecting at least one conformation for the molecule based on at least one parameter related to molecular conformations; and
preparing a report that includes the selected at least one conformation for the molecule.

23. A computer system comprising:
one or more processors; and
one or more non-transitory computer readable media storing instructions that in response to being executed by the one or more processors, cause the computer system to perform operations, the operations comprising:
obtaining molecule graph data for a molecule;
inputting the molecule graph data into a machine learning platform;
generating a plurality of conformations for the molecule with the machine learning platform, wherein the plurality conformations are specific to molecule, each conformation having internal coordinates defining positions of atoms of the molecule;
selecting at least one conformation for the molecule based on at least one parameter related to molecular conformations; and
preparing a report that includes the selected at least one conformation for the molecule.
